(19) United States
(12) Patent Application Publication
(10) Pub. No.: US 2017/0067871 A1
(43) Pub. Date: Mar. 9, 2017

(54) HIGH-THROUGHPUT CELLULAR ANALYSIS USING MICROBUBBLE ARRAYS

(71) Applicant: University of Rochester, Rochester, NY (US)

(72) Inventors: Lisa A. DeLouise, Rochester, NY (US); James J. Kobie, Rochester, NY (US)

(21) Appl. No.: 15/260,270

(22) Filed: Sep. 8, 2016

Related U.S. Application Data
(60) Provisional application No. 62/215,348, filed on Sep. 8, 2015.

(51) Int. Cl. G01N 33/483; B01L 3/00

(52) U.S. Cl. CPC G01N 33/4833; B01L 3/502761; B01L 2200/0652; B01L 2300/0893

(57) ABSTRACT

A microfabricated device and method having a substrate with an array of curvilinear cavities that is used for high throughput single cell screening. The substrate of the device is preferably fabricated in a low elastic modulus polymer such as polydimethylsiloxane. The architecture of the cavity forms a small volume micro-niche that seeded cells can rapidly condition to promote survival and proliferation which can be monitored for hours to days to weeks. The cavity architecture allows independent assays to be conducted with minimal influence from nearest neighbor cavities. Methods are disclosed to use the device to, for example, screen single cells by clonal proliferation, clonal morphology, secreted factors, secretion rate, surface markers, and cell functional characteristics including but not limited to migration, drug resistance, the ability to block or promote signaling pathways, or to enhance opsonization.

This application claims the priority benefit of U.S. Provisional Patent Application Ser. No. 62/215,348, filed Sep. 8, 2015, which is hereby incorporated by reference in its entirety.

### STATEMENT REGARDING FEDERALLY SPONSORED RESEARCH OR DEVELOPMENT

This invention was made with government support under Contract Nos. P30 AI078498, TL1 TR000096, KL2 TR000095, and UL1 TR000042, awarded by National Institutes of Health. The Government has certain rights in the invention.

### FIELD OF THE INVENTION

The present disclosure is directed generally to methods and systems for high throughput single cell screening using a substrate comprising an array of curvilinear cavities.

### BACKGROUND

The ability to sort cells from heterogeneous population and to study them at the single cell level provides unique opportunities for drug discovery, identification of tumor initiating cancer stem cells, and for understanding signaling pathways in disease, among other opportunities. This capability is particularly advantageous for the production of monoclonal antibodies which requires the sorting of potentially rare (1 in >10⁴) antibody producing cells from a heterogeneous population. Monoclonal antibodies (mAb) are a rapidly growing class of human therapeutics with a market size of roughly $78 billion in 2012. Their ability to specifically recognize and bind antigens of interest with high affinity holds vast potential as treatments for diseases ranging from autoimmune disorders to infectious diseases and cancer therapeutics.

Microfabricated technologies have become increasingly popular in cell biology and disease state research due to their ability to capture and monitor single cells in physiologically relevant microenvironments. Within these in vitro microenvironments, heterogeneous cell populations can be sorted and independently interrogated within one device that overcomes many limitations of standard cell culture assay systems. For example, use of the 96 and 386 multi-well plate format imposes the constraint of a high media volume to surface area ratio which hinders cell self-conditioning of wells when seeded under limiting dilution conditions. Relatively large reagent volumes, long processing times (weeks to months), and the necessity to use hundreds to thousands of plates to assay for minority cell types or secreted soluble factors (e.g. cytokines, antibodies) are additional limitations that can be overcome using microfabricated systems. The attributes of a low cell culture volume, customizable surface chemistry, and the ability to fabricate high density micro-well arrays, are particularly advantageous for immune system research in which both single cell studies and interactions between B and T cells can be specifically probed and their secreted products (cytokines and antibodies) can be quantified.

The use of microfabricated devices to quantify immune cell secreted products is particularly useful in the field of monoclonal antibody discovery. Conventional mAb production involves fusing splenocytes from immunized mice with an immortalized myeloma cell line. The resulting hybridoma cells are cultured under limiting dilution conditions (<1 cell per well) in microtiter plates for 7 to 14 days to allow for clonal expansion. The culture supernatants are then tested for antigen specificity using Enzyme Linked Immunosorbent Assay (ELISA) methods to identify the wells containing cells of interest. While this method is effective, the process is laborious, time consuming, and costly. Moreover, relatively few (˜103) of the hybridoma cells produced can be tested and therefore potentially high affinity and/or high efficacy mAbs may be missed.

To expand and simplify the antibody discovery process, microfabrication technologies have been exploited to develop novel single cell high-throughput methods for screening >10⁵ cells from hybridoma cells or screening antigen specific B cells from human peripheral blood. There are several single cell methods reported for detecting antibody secreting cells (ASC) including antigen arrays, droplet based fluidic systems, and micro-well techniques including Microengraving and ISAAC. Microengraving utilizes large arrays of shallow cuboidal micron scale pits formed in polydimethylsioxane (PDMS) to seed cells. The array is capped with a glass slide functionalized to bind secreted mAbs. After ˜2-4 hours in culture the slide is removed from the array, treated with a secondary reporter and then used as a template to locate positive wells containing the cell(s) producing the mAb of interest. The ISSAC technique similarly uses shallow micro-well arrays formed in PDMS to seed cells, however mAb detection is done through direct binding of cell secretions to an antigen specific surface coating. Direct detection of fluorescence around the exterior of a well greatly simplifies the process of locating positive wells. While the aforementioned techniques make vast improvements over the conventional ELISA cell screening process, they still suffer from various drawbacks. In Microengraving, the array capping process limits the nutrient exchange within the shallow picoliter wells and thus limits the time allowed for detecting mAb secretions to only a few hours and therefore only ASC that secrete at a high rate can be detected. While the ISSAC technique does not rely on a cap for signal generation, the open well architecture allows for the loss of cell secretions over time by diffusion and dilution into the bulk media. In shallow well architectures the cells may be easily dislodged by turbulent fluid flow creating uncertainty in being able to recover the specific cell of interest. Neither system allows for clonal expansion of cells which could greatly increase detection sensitivity and thus enable the discovery of potentially high affinity mAbs that are secreted at a low rate as well as recovery of more genetic material for mAb cloning.

Accordingly, there is a continued need in the art for efficient and cost-effective devices, systems, and methods for high-throughput single cell screening and/or analysis.

### SUMMARY OF THE DISCLOSURE

The present disclosure is directed to inventive methods and systems for high throughput single cell screening using a substrate comprising an array of curvilinear cavities. A microfabricated device platform based on microbubble well array technology is utilized for culturing single cells for hours, days, or weeks where they can be sorted based on clonal proliferation, clonal morphology, cell secreted factors, cell function, and other means.

According to one embodiment, microbubble (“MB”) wells are deep (80-250 μm) spherical compartments with 40-100 μm diameter circular openings fabricated in PDMS using the gas expansion molding process. It is shown that the unique MB well architecture facilitates the accumulation of cell secreted factors while allowing for sufficient nutrient and waste exchange to enable cell proliferation. Although sharing similar goals with Microengraving and ISSAC, the MB well architecture and array method takes the technology three critical steps further by: (1) providing an uninhibited niche for cells to proliferate and their secreted factors to concentrate over hours and days without the additional step of capping; (2) simplifying the detection and recovery of cells from wells of interest; and (3) enabling functional assays to be conducted within the MB well microenvironmental niche. The spherical geometry of the MB well allows for the reduction in shear stresses at the base of the MB, which aids in the growth and propagation of the cells, reduces the need for a capping process for cell and secreted factor containment, and minimizes paracrine signaling effects from nearest neighbor wells.

Normally, cells in MB wells are not easily displaced by fluid flow and, in fact, it takes several hours for nonadherent cells to exit the wells of inverted chips. However, according to an embodiment, the interior of the MB wells can be modified to selectively capture cells through antigen specific coatings. Accordingly, described herein are methods and systems utilizing an MB device for conducting high-throughput cell screening assays to identify and recover specific cells in a heterogeneous population based on measuring clonal proliferation, clonal morphology, clonal growth rate, cell secretions, and many other functional assays. In certain embodiments, the device and method are utilized to identify effective drugs for personalized therapeutics. As another example, according to an embodiment the present invention provides a device and method to identify drug resistant cancer cells including cancer stem cells.

In one embodiment, the present invention provides a device and method or screening cells by measuring the rate of secreted factor. Another embodiment of the present invention provides a device and method to sort antigen specific monoclonal and polyclonal antibody by their secretion rates. In certain embodiments, the device and method comprises a means identify antigen specific monoclonal and polyclonal antibody secretions from mouse hybridoma cells, or CHO cells, or B cells from human or animal peripheral blood or lymphoid organs.

In one embodiment, the present invention provides a device and method to sort cells by their secretion rate by quantifying the rate of immunoprecipitation. In another embodiment, the present invention provides a device and method to sort cells by their secretion rate by capturing cell secreted factors onto the surface of the MB well. For example, in one embodiment, the present invention provides a device and method to identify monoclonal and polyclonal antibody secretions by capturing them to the surface of the MB well. Another embodiment of the present invention provides a device and method to identify antigen specific monoclonal and polyclonal antibody secretions by quantifying immunoprecipitates or by capturing them onto the surface of the MB well. In certain embodiments, the device and method comprises a means to identify antigen specific monoclonal and polyclonal antibody secretions from mouse hybridoma cells, or CHO cells, or B cells from human or animal peripheral blood or lymphoid organs.

According to an aspect, a method for analyzing a cell is provided. The method includes the steps of: (1) providing a microfluidic device, the microfluidic device comprising a substrate having: (i) a first surface; and (ii) a plurality of curvilinear cavities embedded in the substrate, each of the plurality of curvilinear cavities comprising an inner surface and an opening at the first surface to an exterior of the substrate, the opening having a first diameter, where the inner surface of each curvilinear cavity curves outward from a rounded bottom located at a point furthest from the opening of the cavity to a maximum diameter, and then curves inward from the maximum diameter to the opening at the first surface, the maximum diameter being greater than the first diameter; (2) adding a plurality of cells to the microfluidic device under conditions configured to allow at least some of the cells to seed within one or more of the plurality of curvilinear cavities embedded in the substrate; (3) incubating the microfluidic device under conditions suitable for the cells to survive for a first period of time; and (4) sorting the cells based on a first characteristic of the cells.

As used herein, the term “microfluidic device” as described above includes a device that during use is intended to contain microscale volumes of fluids, and is not necessarily intended to mean that the device is used for perfusion of fluids through or over surfaces of the device, although both perfusion and static cell culture conditions are contemplated.

According to an embodiment, the plurality of curvilinear cavities embedded in the substrate are configured to form a lattice, and are spaced a predefined distance from each other in order to maintain a substantially homogeneous cavity size within the array regardless of the impact of neighboring wells.

According to an embodiment, the method includes the step of coating the plurality of curvilinear cavities prior to adding the plurality of cells.

According to an embodiment, the coating comprises a chemical, biomolecule, or biochemical. The coating can be, for example, an antibody, a toxin, a growth factor, a selectin, a collagen, a fibronectin, a chemoattractant, a signaling molecule, an antigen, a ligand, a biochemical, and/or combinations thereof.

According to an embodiment, the method includes the step of coating the first surface with a cell or protein blocking agent. The coating can be, for example, bovine serum albumin, casein, polyethylene glycol (PEG), or another blocking agent.

According to an embodiment, the method includes the step of adding a compound to the incubating cells.

According to an embodiment, the method includes the step of incubating the microfluidic device under conditions suitable for the cells to proliferate, where the conditions for survival and the conditions for proliferation may be identical or different.

According to an embodiment, the first characteristic is proliferation, morphology, drug resistance, adhesion, secretion rate, surface marker, ability to block a signaling pathway, ability to promote a signaling pathway, ability to enhance opsonization, and combinations thereof.

According to an embodiment, the method includes the step of visualizing one or more seeded curvilinear cavities.

According to an embodiment, the ratio of the maximum diameter to the first diameter is greater than 1. For example, the maximum diameter can be approximately 88 to 350 microns and the first diameter can be approximately 40 to 200 microns.

According to an embodiment, the cells can be stem cells, cancer cells, mouse hybridoma cells, CHO cells, or B cells derived from human or animal peripheral blood or lymphoid organs. According to an embodiment, the number of cells seeded per curvilinear cavity is controlled.

According to an embodiment, the method includes the step of detecting one or more secretions of the embedded cells. For example, the secretion can be detected with a fluorescently or chromogenic tagged antigen, peptide, cytokine, antibody or other protein or nanoparticle reporter.

According to an embodiment, two or more different cell types are seeded.

According to an embodiment, the substrate comprises a polysiloxane, a polyacrlyamide, a polyacrylate, a polymethacrylate, a carbon-based polymer or mixtures thereof.

According to an aspect, a method for analyzing a cell is provided. The method includes the steps of: (1) providing a microfluidic device comprising a substrate having: (i) a first surface; and (ii) a plurality of curvilinear cavities embedded in the substrate, each of the plurality of curvilinear cavities comprising an inner surface and an opening at the first surface to an exterior of the substrate, the opening having a first diameter, where the inner surface of each curvilinear cavity curves outward from a rounded bottom located at a point furthest from the opening of the cavity to a maximum diameter, and then curves inward from the maximum diameter to the opening at the first surface, the maximum diameter being greater than the first diameter; (2) adding a plurality of cells to the microfluidic device under conditions configured to allow at least some of the cells embed within one or more of the plurality of curvilinear cavities embedded in the substrate; (3) incubating the microfluidic device under conditions suitable for the cells to survive for a first period of time; and (4) analyzing the cells.

These and other aspects of the invention will become clear in the detailed description set forth below.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-B show the heterogeneous morphology of squamous carcinoma cells growing on tissue culture plates, in accordance with an embodiment.

FIG. 2 shows that for a fixed cell stock solution concentration the seeding density (# of cells per MB well) depends on microbubble well opening, in accordance with an embodiment.

FIG. 3 shows the percent of microbubble wells in an array that started with one squamous carcinoma cell to proliferate to >10 cells per well, in accordance with an embodiment.

FIGS. 4A-H show examples of the cell specific clonal morphologies that squamous carcinoma cells produce after five days in culture defined as high positive, sphere and low positive. Clonal morphologies and migratory characteristics in and out of MB wells are cell type dependent, in accordance with an embodiment.

FIG. 5 shows examples of the cell specific clonal morphology and migratory characteristic of primary dermal human fibroblast cells, in accordance with an embodiment. Wells seeded on day 0 with 2 cells do not undergo substantial proliferation in the MB well; rather the cells make matrix and migrate out of the MB well.

FIG. 6 shows an example of cutaneous squamous cell carcinoma cell colonies growing in microbubble wells derived initially from seeding one or two cells per MB well, in accordance with an embodiment. After culturing cells for 7 day the live cells are stained with calceinAM showing distinct proliferation rates and heterogeneous morphologies including spheres, spread cells and cell clusters.

FIG. 7 shows the over clonogenic potential of all MB wells that started with one, two and three squamous carcinoma cells as well as a breakdown of the clonogenic potential of the distinct clonal morphologies as a function of the number of cells seeded per well, in accordance with an embodiment.

FIGS. 8A-F show the life cycle of a squamous carcinoma sphere over 9 days, in accordance with an embodiment.

FIG. 9 shows over all clonogenic potential and sphere formation tendency for three different squamous carcinoma cell lines, in accordance with an embodiment. A higher tendency to form spheroids decreases over all clonogenic potential as defined.

FIG. 10A-B shows that primary human keratinocytes cultured in uncoated MB wells for 8 days do not grow thus allowing differentiation between cancerous and noncancerous skin cells, in accordance with an embodiment.

FIGS. 11A-C show immunofluorescence stain for cytokeratins CK5 (basal) and CK10 (differentiated) on squamous carcinoma cells cultured in microbubble wells for 9 days where cells have proliferated and begun to migrate out of the well and to spread out onto the chip planar surface around the opening of the microbubble well, in accordance with an embodiment. Cells predominately stain for CK5 although some punctate cells in MB wells stain for CK10. These cells likely differentiated and died.

FIG. 12 shows immunofluorescent stem cell marker staining of drug resistant squamous cell carcinoma sphere cells, in accordance with an embodiment.

FIGS. 13A-C show an array of cutaneous squamous cell carcinoma cell colonies growing in microbubble wells derived initially from seeding one or two cells per MB well before (13A) and after (13B) culturing the cells in 10 μM Cisplatin for 48 hr and treating with CalceinAM to stain live cells (green) and propidium iodide to stain dead cells (red), in accordance with an embodiment. Results show spheroids are drug resistant tumor initiating cancer stem cells. FIG. 13C shows zoomed in view of surviving sphere cells that comprise ˜5% of initial cells seeded in the MB well array.

FIGS. 14A-B show regions of a MB well array with established colonies of squamous carcinoma cells on Day 5 before and after treatment with cisplatin showing sphere cells exhibit drug resistance as they stain positive with calceinAM, in accordance with an embodiment.

FIG. 15 shows a close up view of drug resistant sphere cells for two squamous cell carcinoma cell lines, in accordance with an embodiment.

FIG. 16 shows immunofluorescent stem cell and cytokeratin CK5 marker staining of drug resistant squamous cell carcinoma sphere cells, in accordance with an embodiment.

FIGS. 17A-F show example of plucking cells using micromanipulator tools from a MB well and transferring them to a tissue culture plate, in accordance with an embodiment.

FIGS. 18A-B show the variation in A375 melanoma cell morphology when seeded in MB wells, in accordance with an embodiment.

FIGS. 19A-B show results from radial migration assay from MB wells for melanoma cell line that vary in metastatic potential, in accordance with an embodiment.

FIG. 20 shows a quantitative comparison of size dependent microbubble well parameters, in accordance with an embodiment.

FIG. 21 shows that adjusting the cell stock concentration equivalent seeding distribution in different size MB wells can be achieved, in accordance with an embodiment.

FIG. 22 shows that the clonogenic potential depends on the number of cells seeded per well as well as the size of the MB well, in accordance with an embodiment.

FIG. 23 shows that detection of secreted factors depends on the size of the MB well; signal is detected earlier in smaller well, in accordance with an embodiment.

FIG. 24 shows a bar graph quantifying the % of wells that are positive for IgG secretion over time as a function of microbubble well size, in accordance with an embodiment.

FIG. 25 shows a graph of the net fluorescent intensity values for 59 individual 200 micron MB wells over the course of 5 days of incubation, in accordance with an embodiment.

FIG. 26 shows a graph of the net fluorescent intensity values for 142 individual 100 micron MB wells over the course of 5 days of incubation, in accordance with an embodiment.

FIG. 27 shows a graph of the net fluorescent intensity values for 224 individual 60 micron MB wells over the course of 5 days of incubation, in accordance with an embodiment.

FIG. 28 shows a section of a microbubble well array cultured over time initially seeded with one or two antibody secreting cells per well thus allowing sorting of cells by secretion rate, in accordance with an embodiment.

FIGS. 29A-B show results of a functional assay conducted in a microbubble array allowing selection of B cells that produce highly efficient bacterial opsonization by macrophage cells, in accordance with an embodiment.

FIGS. 30A-B illustrate the analyses of single cell seeding for clonogenic potential assay. FIG. 30A shows Box and Whisker graphical representation of the positive fractions of single cell seeded wells that showed clonal expansion in culture by Day 5. The top and bottom of the boxes show the upper and lower 25 percentiles, the band in the middle of the box is the median and the bars at the upper and lower end represent the minimum and maximum values. A student's t-test with a confidence level of 0.05, the **p<<0.001, n=73, 90, and 89 single cell cultures counted for the A375 P, A375 MA1, and A375 MA2 cell lines, respectively. FIG. 30B shows the distribution of single cell proliferation outcomes; a single cell expanding to a sphere (→1s), 2 cells (→2), and ≧3 (→3) or cell death (→0) and no growth (→1).

### DETAILED DESCRIPTION

The present invention provides microfabricated device and methods for conducting high throughput cellular screening. The microfabricated device is comprised of an array of curvilinear cavities fabricated in a low elastic modulus polymer such as polydimethylsiloxane that provides a microenvironmental niche to culture cells with a modulus similar to soft tissue. The methods are described using this device to conduct single cell high throughput screens of heterogeneous cell samples. Single cells can be sorted by clonal proliferation, clonal morphology, cell adhesion, secreted factors, secretion rate, surface markers, and cell functional characteristics including but not limited to the ability to block or promote signaling pathways, or to enhance opsonization.

### The Microbubble (“MB”) Well Array

Microbubble well arrays can be utilized, for example, for single cell sorting using microbubble well arrays. According to an embodiment, the microbubble well array is a substrate comprising a first surface and a plurality of curvilinear spherical cavities embedded in the substrate. Each of the curvilinear spherical cavities has an inner surface and an opening, with a first diameter, at the first surface to an exterior of the substrate. The inner surface of each curvilinear spherical cavity curves outward from a rounded bottom located at a point furthest from the opening of the cavity to a maximum diameter, and then curves inward from the maximum diameter to the opening at the first surface. The maximum diameter is greater than the first diameter. According to an embodiment, the ratio of the maximum diameter to the first diameter is greater than one (1). For example, the maximum diameter may be approximately 80 to 350 microns, and the first diameter may be approximately 40 to 200 microns. According to an embodiment, the cavities are spaced at a distance in a range of about two times the diameter of the opening of the cavities to about ten times the diameter of the opening of the cavities, although other configurations are possible.

According to an embodiment, the substrate can be, for example, a polymer such as a polysiloxane, a carbon-based polymer, PDMS, a polyacrlyamide, a polyacrylate, a polymethacrylate, or mixtures thereof, among many other polymers.

According to an embodiment, the curvilinear spherical cavities can be provided in an array with the cavities arranged in evenly spaced rows, in staggered rows, with cavities of the same size, cavities of the same shape, and/or cavities of varied sizes, among other variations.

According to an embodiment, the curvilinear cavities embedded in the substrate can be configured to form a lattice structure. Additionally, the curvilinear cavities can be spaced a predefined distance from each other in order to maintain a substantially homogeneous cavity size within the array regardless of the impact of neighboring wells.

According to one embodiment, the MB well arrays are formed in PDMS using a gas expansion molding (GEM) process. Fabrication of the MB array can be carried out using the procedures described in Giang et al., “Microfabrication of Bubbular Cavities in PDMS for Cell Sorting and Microcell Culture Applications,” J Bionic Eng 5(4):308e16 (2008); Giang et al., “Micro fabrication of Cavities in Polydimethylsiloxane Using DRIE Silicon Molds,” Lab on a Chip 7:1660-1662 (2007); and PCT Application Publ. No. WO 2008/157480, each of which is hereby incorporated by reference in its entirety. Briefly, the process utilizes a silicon wafer mold that contains an array of cylindrical pits of approximately 40 to 200 μm in diameter and approximately 20 to 300 μm in depth, where 150 μm in depth is preferred, with each feature being spaced 4× apart from one another on a square lattice. These features can be etched into the wafer utilizing the Bosch deep reactive ion etch process (Plasma Therm 770, MEMS and Nanotechnology Exchange LLC, Reston, Va.). To cast the PDMS MB well array, a 10:1 base to elastomer curing agent ratio was used (Sylgard, Dow Corning, USA). The prepolymer can be mechanically mixed in a 50 mL conical tube with a plastic pipette for approximately 20-30 seconds or until adequate mixing is achieved, as noticed by the formation of air bubbles within the suspension. The PDMS premix is then poured onto the silicon wafer to achieve a PDMS thickness of ˜2 mm. The PDMS is left to self-level at room temperature for ˜10 minutes. Following the self-leveling process the residual bubbles left in the PDMS layer may be removed by mechanical puncturing with a sterile pipette tip. The silicon wafer with the PDMS premix is then moved to a ˜100° C. oven to cure for 1 hour. A spherical MB well forms over each deep pit in the silicon wafer. The size of the MB wells produced have circular opening of about 40 to about 200 μm in diameter and overall diameter of about 80 to about 300 μm, but preferably about 40 μm in diameter with overall diameter of about 120 μm. Following the MB well formation process, the wafer and the PDMS cast are removed from the oven and the PDMS cast is peeled away from the silicon wafer mold. The MB array can then be cut to a desired size using a surgical blade and stored at room temperature in an enclosed Petri dish for future experiments. This process, and variations thereof, are fully described in U.S. patent application Ser. Nos. 13/469,184 and 13/304,843 to DeLouise, each of which is hereby incorporated by reference in its entirety.

### Cell Seeding of the Microbubble Array

In one embodiment, the present invention includes a single cell screening method whereby cells are seeded into the microbubble well arrays.

Any number of cell types can be cultured in a WB well array in accordance with the methods disclosed herein. These cell types include cell lines and primary cell isolates, as well as secondary cell cultures derived therefrom.

Exemplary types of cell lines include immortalized cell lines, as well as cancerous and non-cancerous cell lines.

Non-limiting examples of immortalized cell lines include, without limitation, mouse 3T3 cells, rat F11 neuronal cells, human HEK293 cells, monkey Vero kidney cells, and any hybridoma cell lines. Other immortalized cell lines can also be used.

Non-limiting examples of cancer cell lines include, without limitation, the cervical cancer cell lines HeLa, SiHa, Ca Ski, and C-33A; the breast cancer cell lines 600MPE, AU565, BT-20, BT-474, BT-483, BT-549, Evsa-T, Hs578T, MCF-7, MDA-MB-231, SkBr3, and T-47D; the non-small cell lung cancer cell lines A549, EKVX, HOP-62, HOP-92, NCI-H226, NCI-H23, NCI-H322M, NCI-H460, and NCI-H522; the colon cancer cell lines COLO 205, HCC-2998, HCT-116, HCT-15, HT29, KM12, and SW-620; the CNS cancer cell lines SF-268, SF-295, SF-539, SNB-19, SNB-75, and U251; the melanoma cell lines LOX IMVI, MALME-3M, M14, MDA-MB-435, SK-MEL-2, SK-MEL-28, SK-MEL-5, UACC-257, and UACC-62; the leukemia cell lines CCRF-CEM, HL-60(TB), K-562, MOLT-4, RPMI-8226, Jurkat and SR; the ovarian cancer cell lines IGR-OV1, OVCAR-3, OVCAR-4, OVCAR-5, OVCAR-8, NCl/ADR-RES, and SK-OV-3; the renal cancer cell lines 786-0, A498, ACHN, CAKI-1, RXF 393, SN12C, TK-10, and UO-31; the prostate cancer cell lines PC-3 and DU-145; and the glioma cell lines SW1783, SF767, SF-767, SF-763, A-172, U-87 MG, U-251 MG, U-343 MG, and SF-539. Other cancer cell lines can also be used.

Non-limiting examples of non-cancerous cell lines include, without limitation, HEL 299, MRC-5, IMR-90, CCD-19Lu, MRC-9, and WI-38 human lung fibroblasts; BEAS-2B, NL20, and NL20-TA human lung epithelial cells; and NR8383 human macrophages. Other non-cancerous cell lines can also be used.

Primary isolates can include those cell types derived from soft tissue biopsies, solid tumor biopsies, bone marrow biopsies, and any other forms of tissue biopsy, as well as cell types obtainable by tissue sampling procedures such as swabbing or scraping a tissue surface with an appropriate sample collection device, and cell types obtainable by blood, serum, or other body fluid sample collection procedures. If desired, primary cell isolates can be sorted prior to culturing in a MB array using flow cytometry or other means. As such, it is possible to introduce a pre-sorted (or substantially homogeneous) population of primary cell isolates onto the MB well array. Alternatively, a heterogeneous population of primary cell isolates can be introduced onto the MB well array with little or no pre-sorting of the cells present in the various types of biopsies or cell samples as identified above.

To prepare the arrays for cell seeding, the MB well array chips are placed on a glass slide with the MB well openings facing down. The slide and the arrays are then treated for 60-90 seconds in oxygen plasma to increase the backside chip wettability (March Instruments Inc., USA). Other approaches for improving wettability can also be used. Following the plasma treatment, the chips are moved into separate wells of a 24 well plate and submerged in 1 mL of 1:1 DI water and ethanol. The 24 well plate containing the MB arrays is then placed in a vacuum chamber and degassed for 2-3 minutes or until the MB array becomes clear, signifying successful priming (infiltration of liquid into the MB wells) of the arrays. This step also serves to sterilize the chip before cell seeding. The MB arrays are then moved to new wells of the 24 well plate and submerged in 1 mL of 1× PBS (BP13351 Fisher BioReagents, USA) under sterile conditions. The 24 well plate with the MBs is then moved to a 37° C. incubator where they were stored for a minimum of 20 hours. Following incubation in PBS, chips can be moved to new wells on the 24 well plate and incubated in fresh media (e.g., RPMI 1640, Gibco A10491-01, Invitrogen Corp., USA) for 10-15 minutes at 37° C. to allow for priming of the MBs with media. The chips are then moved into new wells and 1 mL of cell suspension is pipetted into the wells at a seeding density of ˜15,000 cells/cm² in order to achieve efficient single cell capture. Chips are incubated for approximately 10 minutes before being rinsed and placed into new wells containing fresh media (e.g., RPMI 1640, Gibco A10491-01, Invitrogen Corp., USA). Previous studies quantified how the MB well array seeding statistics (% of wells with 0, 1, 2, 3 etc. cells/well) depends on the cell stock concentration, incubation time, and the MB well opening size. The seeding statistics for the conditions described above produces arrays with 62%±5% of wells with 0 cells, 28%±2% of wells with 1 cells, 7%±4% of wells with 2 cells, 2%±2% of wells with 3 cells, and 0.3%±0.6% of wells with ≧4 cells/well. Using higher or lower seeding densities will, of course, alter the frequency of wells having no cells, 1 cell, or more than 1 cell per well.

According to another embodiment, the cells can be seeded using one or more other methods once the MB chips are prepared for cell seeding using any of the methods described or otherwise envisioned herein. For example, according to one embodiment the following method is utilized to seed fixed MB chips. Prior to seeding, the cells are prepped. A live cell count is done using a 1:1 trypan blue dye and prepared cell suspension. The cells are then counted on a cell counter (TC10™ Automated Cell Counter, BioRad) to determine the concentration of live cells present in the solution. Using this live cell count, a dilution is calculated to prepare the desired cell seeding concentration. For most cells seeded into 60 μm MB openings, a live cell density of 15,000 cells/cm² or 30,000 cells/mL for 1 mL in a 24-well plate can be used for MB cell seeding. This should result in a seeding distribution of approximately 48% of the MBs containing 0 cells, 35% containing 1 cell, 10% containing 2 cells, and 7% containing 3 or more cells after incubating for ˜8 minutes. The cell seeding density can be altered depending on the cell type, settling characteristics, the size of the MB opening diameter, and/or the incubation time with the cell solution during seeding, among other variations.

The cells are then seeded into the MBs by removing the media from the chips, being extra careful as to not deprime the MBs, and pipetting the appropriate cell density dilution over the MB chip. Once seeded, the MB chips are incubated in an incubator at 37° C. and 5% CO₂ for about 8-10 minutes to allow the cells to settle into the MBs, which can be observed under a microscope. For some cells, a longer incubation time may be needed to account for the variation in cell settling times. This media containing the cell dilution is then aspirated from the well and the well rinsed thoroughly, about three to four times, with fresh media to clear away any cells left behind on the surface of the MB chip. The chips can then be incubated with fresh media and a count of the cells seeded in each MB of the arrays is performed immediately for indexing to determine the cell seeding statistics. After recording the number of seeded cells per MB, fluorophore-conjugated secondary antibody for detection is added to the TCP well at a concentration of about 0.002-0.004 μg/uL in cell culture media. The concentration of detection antibody used depends on the system and what cell-secreted factor is being analyzed. This is then allowed to incubate for a desired amount of time and can then be imaged to track fluorescent ring development for detection of cells secreting the desired factor within the MBs. Furthermore, the cells can be plucked from individual MBs using CellTram® and InjectMan® NI 2 micromanipulator (Eppendorf) and resuspended in a new location for further expansion and culture of a clonally pure cell line for further analysis.

According to another embodiment, the cell seeding method takes into account a scenario where there is an inability of the MB chips to be physically moved between wells. In some embodiments, the seeding protocol involves picking up the MB chips with tweezers and moving them to different wells on a TCP for the coating steps, the priming of the MBs, the cell seeding, rinses after seeding, and final incubation. Also, during the rinse step after seeding, the MB chips can be shaken back and forth in fresh media in the well of a TCP, and then titled sideways to clear any free cells from the chip surface. In an embodiment where chips are fixed, a bench top platform rocker table (VWR Rocking Platform 200) can be set to a moderate speed setting as means to shake the cells clear from the MB chip surface during the rinsing step. Compared to an embodiment where rocking is not utilized during rinsing, the rocking method during rinsing typically results in higher cell seeding densities, with some MBs containing up to 6 cells per MB. This is due to the rocking motion of the platform causing the cells on the chip surface to move back and forth with the fluid motion. The cells are able to come into contact with more MB openings and fall into the MBs, resulting in MBs with a higher number of cells seeded. In contrast, the rinsing method that does not use rocking typically results in better single cell seeding (˜35% of the MBs contain one cell) and less MBs with multiple cells per MB (less than 30% of the MBs contain two or more cells). For single cell seeding densities, this cell per MB distribution achieved without the rocker table is more highly desired and, thus, the rocking method can be avoided for this target cell/MB density.

Regardless of the specific seeding procedure employed, once the MBs are seeded, the cells therein can be grown in the presence of appropriate media and/or agents for any of a variety of periods of time. In particular, cells can be grown for a period of hours, days, or even weeks, using appropriate changes of media, if necessary.

Cells grown on the MB arrays can be analyzed according to the procedures described herein using appropriate detection reagents (e.g., labeled reagents or dyes), and then selected for further analysis or recovered for subsequent culturing or proliferation under standard tissue culture conditions or MB array culturing conditions of the types described herein.

### Sorting Single Cells by Clonal Proliferation, Morphology and Drug Resistance

In one embodiment, the present invention describes a single cell screening method using microbubble well arrays where single cells can be sorted by clonal proliferation and morphology. Microbubble wells have been used to grow arrays of homogeneously sized microtumors, to study the epithelial-mesenchymal-like transition process that is important in cancer metastasis, and to determine the clonogenic potential of samples enriched with cancer stem cells. Furthermore, a microbubble well array based perfusion system utilized for culturing multi-cellular tumor spheroids reveals that spheroid cells are more resistant to doxorubicin treatment than adherent cells.

Tumor composed of heterogeneous population of both cancer and stromal cells. Microbubble well arrays can be utilized to dissect the cellular heterogeneity at the single cell level which is important for sorting tumor initiating cancer stem cells. Tumor-initiating cells (TICs) are believed to be the distinct subset of tumor cells that have the ability to self-renew, resist chemotherapy and initiate tumor metastasis. Isolating and characterize TICs would provide opportunities to develop much more targeted chemo therapies against metastasis. Microbubble array technology provides a novel platform for identification and characterization of heterogeneous cell populations including TICs.

The results shown herein have demonstrated the utility of MBs are not only a cell culture device, but also a platform for dissecting intrinsic cellular heterogeneity of tumor samples to evaluate its proliferative potential and to develop tumorigenic markers. Accordingly, the kinetics of microenvironment conditioning in a MB well can depend on, for example, the cell type, the number of cells seeded per well, the MB volume, and the MB surface coating. Using simple cell culture techniques and a simple, inexpensive reporting system, MB array technology has proven useful in characterize distinct cell types. More importantly, morphologically distinct clones can be recovered and tumorigenicity characterized using MBs. With further optimization of the reported methods, a comprehensive high-throughput screening assay can be made available to develop prognostic and predictive biomarkers and for advancing basic understanding of cancer metastases.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Sorting Tumor Initiating Cells by Clonal Proliferation and Morphology

Skin cancers comprise a large public health concern and an increasing burden on the health care system. Among 2 million diagnosed skin cancers annually in the U.S., ˜ 35% are squamous cell carcinomas (SCCs). SCC metastases account for the majority of non-melanoma skin cancer deaths. Tumor size and morphology are considered to be prognostic factors for recurrence, but there are no genetic mutations or predictive biomarkers that can be reliably use to identify tumor aggressiveness at an early stage. The characterization and quantification of cells with tumor initiating capability is essential for developing prognostic biomarkers and for advancing the basic understanding of metastases. Current methods of identifying tumor-initiating cells (TICs) are using flow cytometry and serial in vivo xenotransplantation models. However, the cell surface markers derived from these methods failed to identify consistent expression patterns between tumorigenic and non-tumorigenic cell samples.

According to an embodiment, the methods and systems described or otherwise envisioned herein are utilized to quantify clonogenic potential (“CP”) of aggressive SCC tumor cell samples and to recover and characterize TIC clones. The methods and systems take advantage of the fact that TICs have a privileged capacity to condition its microenvironment to survive and proliferate. The unique architecture of the PDMS MB well can facilitate their identification and characterization. Using the high throughput screening capability of the MB well array, the heterogeneity of the SCC tumor cells is dissected at the single cell level to evaluate cellular proliferative potential and tumorigenic markers. Morphologic, MB migratory characteristics, and CP value of well-defined cell samples were studied, including tumorigenic SCC cell lines, non-tumorigenic primary keratinocytes (PKC) samples and primary dermal fibroblasts (FB) cell lines. The results demonstrate that the tumorigenic SCC cells do proliferate in uncoated MB wells and proliferative clones derived from a single cell exhibit differences in growth rate and colony morphology. FB and PKC exhibits distinct morphologic and migratory characteristics from SCC cells. The MB assay and micromanipulation tools can be used to recover and culture cells from different morphologies to test for tumorigenicity.

### Cell Cultures

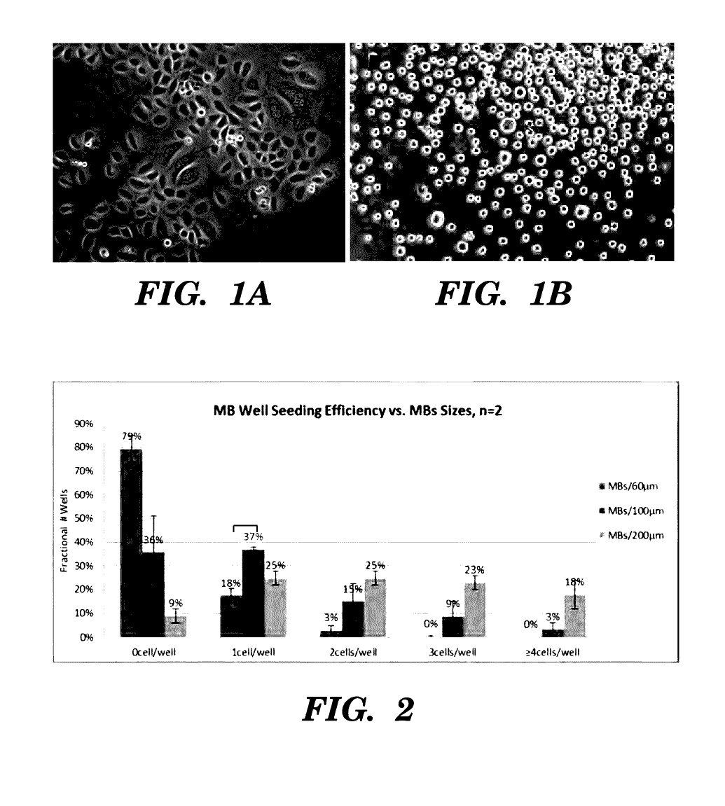

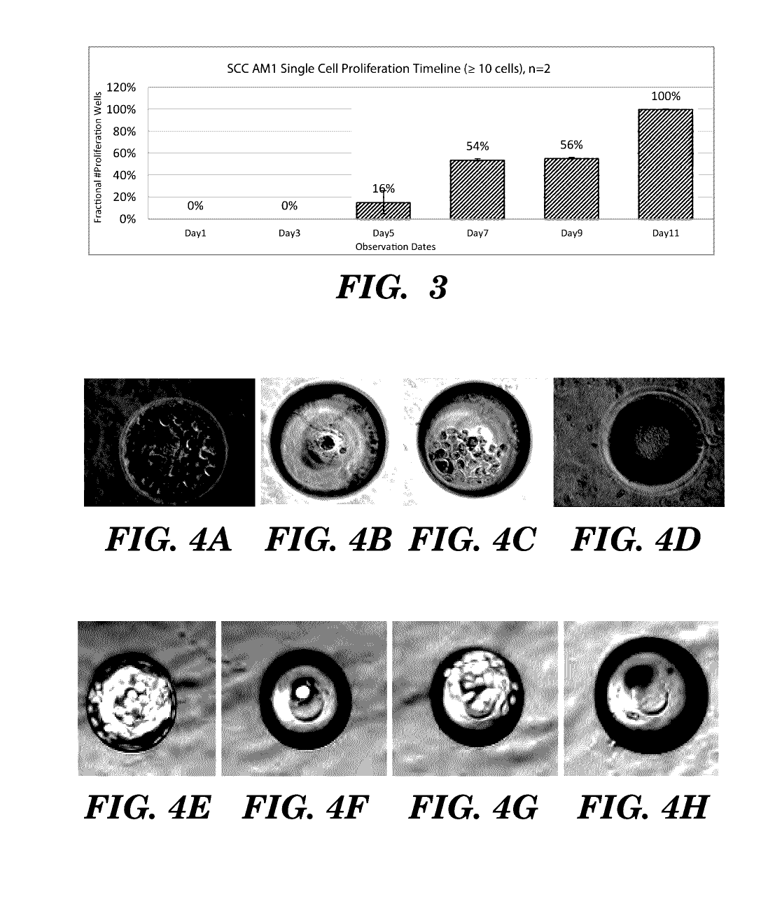

Tumorigenic SCCAM1, AM2, and AM3 cutaneous cell line (cultured in KGM) are derived and characterized at University of Rochester. They express keratin 5 (marker of basal keratinocytes), and were used to study clonogenic potential (CP) and colony morphologies. MB array results are being correlated with SCCAM1 cells cultured on 2D plates which are observed morphologically to be comprised of distinct cell types (FIG. 1). Three non-tumorigenic primary keratinocytes (PKC) samples (cultured in KGM) and two different primary dermal fibroblasts (FB) cell lines (cultured in DMEM/FBS) derived from ex vivo human skin were used to aid in the analysis of tumor samples. The tumorigenic SCC cell lines contains TICs ensuring the ability to observe their characteristics and to distinguish differences between PKC and FB cells which are likely to be present in SCC tumor derived samples.

### PDMS Microbubble Arrays

PDMS MB arrays were prepared using the procedures described previously (see Giang et al., “Microfabrication of Bubbular Cavities in PDMS for Cell Sorting and Microcell Culture Applications,” J Bionic Eng 5(4):308e16 (2008); Giang et al., “Micro fabrication of Cavities in Polydimethylsiloxane Using DRIE Silicon Molds,” Lab on a Chip 7:1660-1662 (2007); PCT Application Publ. No. WO 2008/157480, and U.S. patent application Ser. Nos. 13/469,184 and 13/304,843 to DeLouise, each of which is hereby incorporated by reference in its entirety). Briefly, a 10:1 ratio mixture of base to curing agent (wt %) PDMS was poured over a silicon wafer mold with a hydrophobic coating and cured at 100° C. for 2 hours. Hydrophilic surface modification was conducted using RF Plasma techniques. In this example, chips were utilized that have 100 uncoated, hydrophobic MBs with ˜100 μm diameter circular opening. Each MB chip was placed in a well of a 24 well plate (2 cm²). The planar surface of the MB chips was blocked with 2% BSA for 45 min. Chips were washed one time with 1× PBS, followed by de-priming the MBs in a mixture of 1:1 ratio of PBS and ethanol. The MBs chips were then incubated in PBS overnight before seeding.

### Single Cell Seeding and Live Staining

Results of the MB well seeding efficiency (#cells/well) for a fixed cell seeding density (0.5×10⁴ cells/cm²), shown in FIG. 2, demonstrate a strong positive correlation with MBs diameter circular opening. The larger opening allows more cells to deposit into the well. Tumorigenic SCCAM1, AM2, AM3 were seeded onto MB arrays with controlled seeding densities. A key property of the MB well architecture that is exploited in the CP assay is the low culture media volume (8 nl) per cell ratio. The density of cell stock solution was controlled for seeding MBs ˜0.5×10⁴ cells/cm² (˜1×10⁴ cells/ml). The chips were submerged in the cell stock solution for ˜5 min, and were shaken and washed twice with media to release cells that still deposit onto the planar chip surface. The MBs chips were then placed in a well of a 24 well plate with media. Only the cells in the MBs were counted on day 0. Media was changed every 2 days. This seeding protocol ensured a total seeding efficiency of 80.1%±6.2%, single cell seeding efficiency of 31.3%±4.9%, and ˜170 cells seeded per chip (1.7 cells/well). See FIG. 2.

Clonogenic potential (CP) is defined as the ability of a single cell to generate daughter cells or to self-renew. Cells were cultured in MBs for up to 7 days and CP values were scored after staining with CalceinAM, a widely used reagent for determining cell viability. CP value is determined based on morphologic and MB migratory characteristics. Clonogenic Potential (CP) studies were conducted with the diameter=100 μm (˜8 nL MB volume) well arrays. Images were taken under bright field and fluorescent filter, and were analyzed using ImageJ. One-way Anova test was performed to assess statistical significance between samples (≧3).

After seeding SCC cells in MBs wells they begin to proliferate over time and at some point the cells will migrate out of the well and continue to proliferate on the planar surface of the chip. The percent of wells in an array out of which SCC cells migrate out of the well as a function of time was tracked. See FIG. 3. This migratory assay can be used as a metric to characterize metastatic potential of cancer cells since more aggressive cells should migrate more readily than those with low/no metastatic potential. The result suggests that days 5 to 7 are the best for quantifying CP.

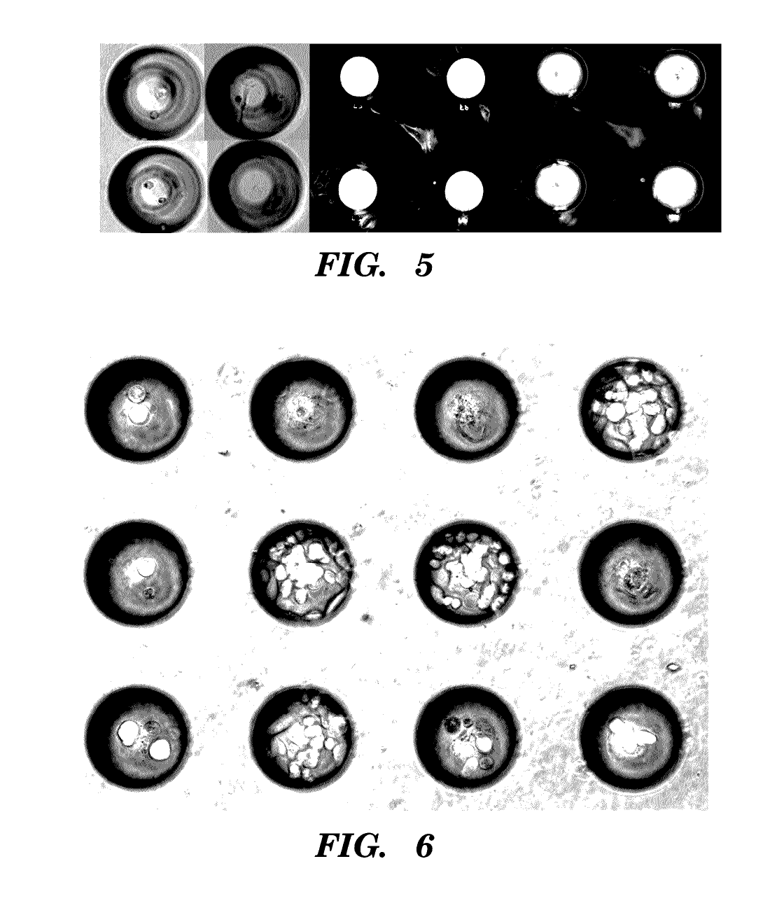

Morphologies of clonal cell cultures for SCC, pKCs and FBs are enumerated in FIGS. 4 and 5. The proliferating clones derived from single cells exhibit differences in growth rates and colony morphologies, as shown in FIGS. 4 and 6. The majority of wells contain cells with spread morphology similar to that observed on tissue culture plastic (see FIGS. 4A, 4C, 4E, 4G, 4H), the remainder (25.5%±4.8% SE) of proliferative colonies derived from a single cell exhibit a sphere/clustered morphology (see FIGS. 4B, 4F).

Studies suggest that cancer cells grown as spheroids are more aggressive and often more tumorigenic and drug resistant than their adherent counterparts. It is also known that in vivo cancer stem cells reside in tissue niches as slow-cycling quiescent cells. In situ studies of stem cell surface markers on the slow growing sphere cells as well as conducting functional assays including invasion, tumorigenic and genomic profiling can prove that these are aggressive tumor initiating.

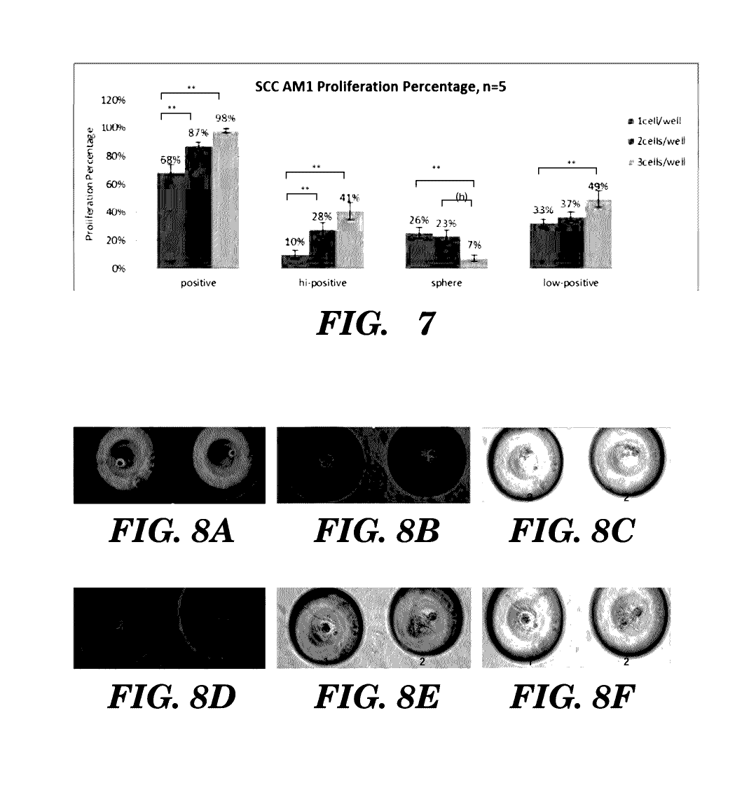

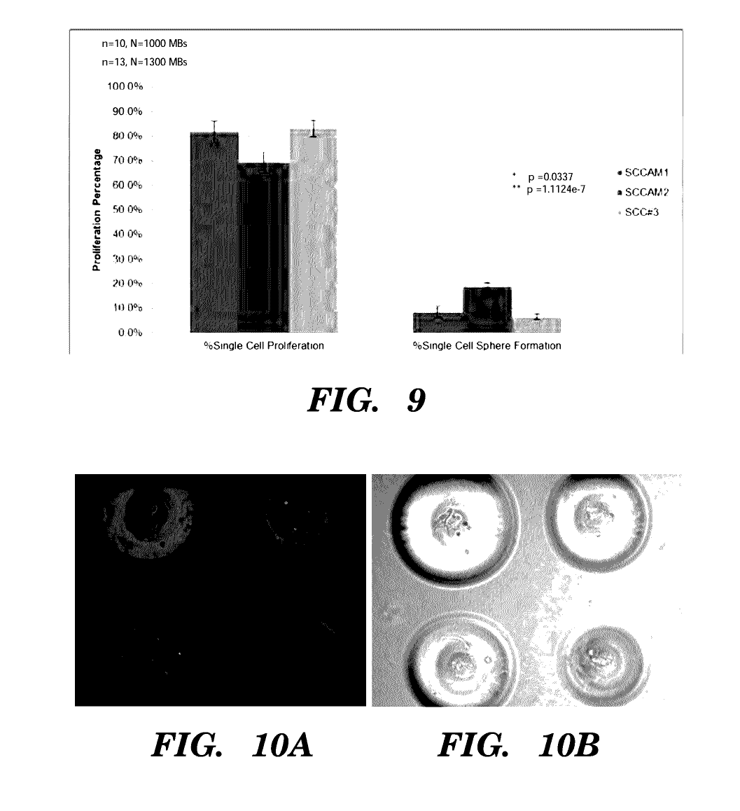

The correlation of a higher CP value with the sphere-forming subpopulation suggests the ability of MB assay to differentiate cell samples that vary in tumorigenicity, as many studies report that cells proliferating as spheres are more tumorigenic. Experimental results of SCC cell lines shows 68.3±5.6% SE (n=9 separate 10×10 microbubble array studies) of the wells seeded with a single cell go on to proliferate in uncoated MBs, which proves that SCC cells possess a privileged capacity to survive in uncoated MBs (see FIG. 7). It was observed that when more cells were initially seeded in a single well, there was a higher possibility of proliferation (positive well) later on whereas when fewer cells were initially seeded in a single well, there was a higher possibility of sphere formation. This reaffirms the significance of single cell seeding for studying the correlation between sphere-forming subpopulation and tumorigenicity. It was also discovered that cells proliferate as spheres form spheroids at an early stage (see FIG. 8). One-way Anova test shows significant differences in single cell proliferation rate and sphere formation rate between distinct cell lines (see FIG. 9). These results demonstrate successful application of MB array assay to analyze tumorigenic SCC cell lines.

By comparison, fibroblasts adapt to the uncoated PDMS, then stretch across the diameter of the MB well and migrate out of the well (see FIG. 5). This indicates that FBs can be distinguished morphologically from SCC cells. No MB wells with proliferating PKC cells were observed 8-10 days post seeding, which suggests that non-tumorigenic primary keratinocytes may not proliferate in uncoated MBs. This will allow for sorting of tumorigenic and non-tumorigenic cells (see FIGS. 10A-10B).

### Example 2

### SCC Cytokeratin Gene Expression

Stem cells are immature cells and have the ability to develop into different cell types. To test whether SCC cells cultured in MBs express cytokeratin 5, validating a basal proliferative SCC phenotype, the expression of cytokeratin 5 and 10 was investigated. Briefly, a MB chip array was placed in 24-well TCP and then transferred into wells containing 1× PBS. It was then transferred into wells containing 10% formalin fixative with 0.1% Triton for 20 min at room temperature. The chip was rinsed twice with 1× PBS, and blocked with 2% BSA for 30 min to prevent any non-specific adsorption of antibodies. A mixture of primary keratin 5 and keratin 10 antibodies (500 μl of 1:500 dilution) in 2% BSA was pipetted onto the top of the chip. The TCP was placed in 4° C. fridge overnight in the dark. Then, the excess antibody solution was removed the next day and the chip was rinsed twice with 1× PBS. The TCP was placed on a shaker for 5 min. A mixture of secondary antibodies Texas Red k5 and FITC k10 (500 μl of 1:500 dilution) in 2% BSA was pipetted onto the top of the chip. The TCP was incubated for 60 min. Then the excess antibody solution was removed and the chip was rinsed twice again with 1× PBS. The chip was stored in 2% BSA and imaged using a fluorescent microscope.

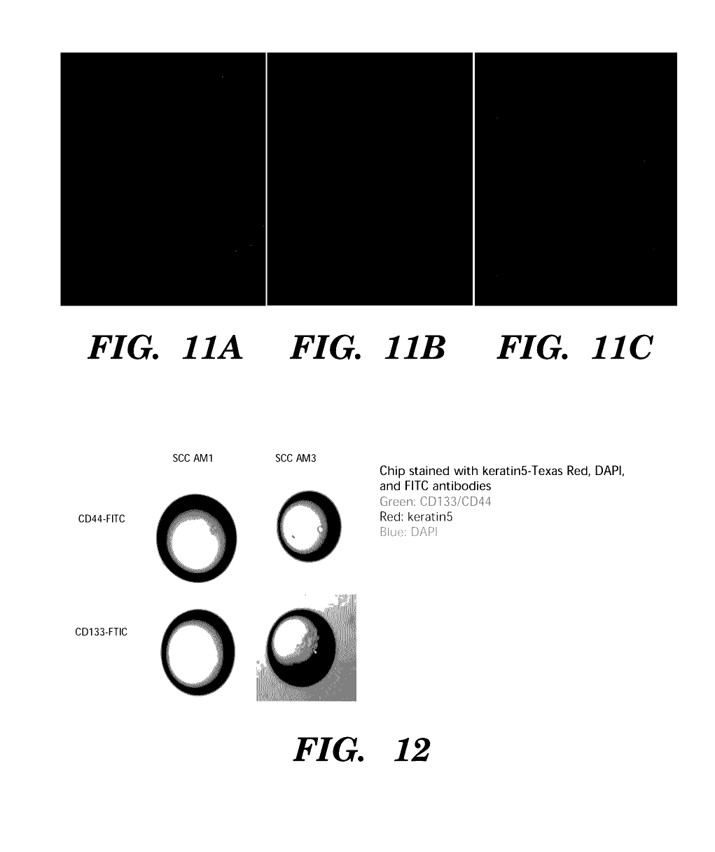

Results show that the SCCAM1 cells cultured in MBs are characterized by cytokeratin 5 expression, validating a basal proliferative SCC phenotype (see FIGS. 11A-11C). The results also show (see FIG. 12) that spheroid cells in the SCC cell population also appear to be K5 positive. Although SCC AM1, 2, and 3 cell lines are tumorigenic, the frequencies of tumorigenic cells are unknown. Based on recent studies it was expected that the frequency of TICs would range between 1:4000 and 1:40000, which are within the range of the ability to detect using small arrays with ˜2500 wells. The cell recovery protocol is being optimized using protocol micromanipulation tools for different cell types, and experiments will be conducted to culture cells from the spread and sphere morphologies to test for tumorigenicity. Moreover, recovered cells will be proliferated using MB arrays or other cell cultures while monitoring the recovered and proliferated cells for maintenance of their phenotype.

### Example 3

### Identification of Tumor Initiating Cells by Drug Resistance

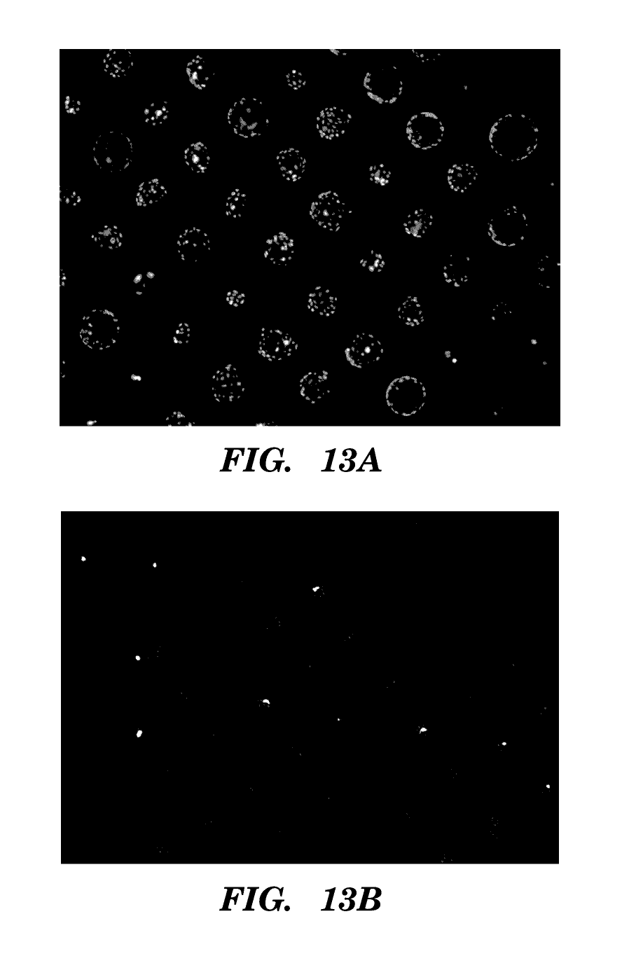

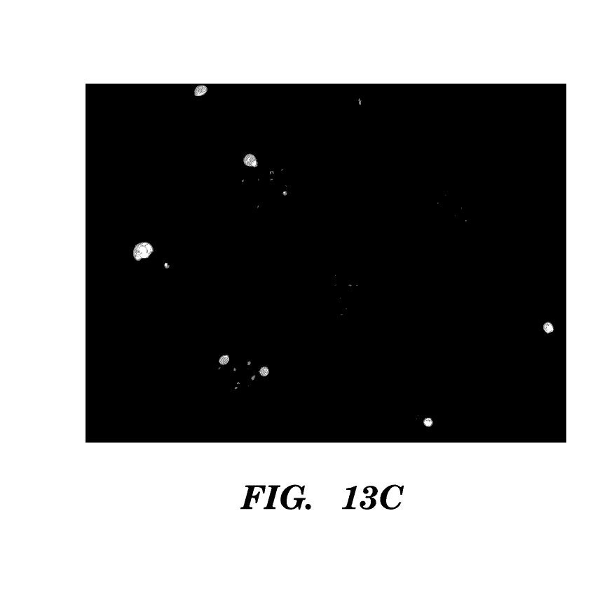

According to an embodiment is a single-cell screening method using microbubble well arrays where morphologically distinct clone can be sorted by drug resistance. Genetic mutations in cancer cells give rise to their aggressive phenotype and their ability to proliferate in a nonadherent fashion with reduced drug sensitivity, which are hallmarks cancer stem cells. Seeding and establishing clonally pure cancer cell colonies in microbubble well arrays is a means to screen for drug resistance as shown in FIGS. 13A-C, 14A-B, and 15. It is shown that the spheroid cell populations are drug resistant and may be representative of tumor initiating cells or cancer stem cells. To enrich for the sphere cells, established colonies were cultured in the presence of a chemotherapeutic agent (e.g. Cisplatin). Traditional chemotherapeutic agents work by killing rapidly dividing cells, one of the main properties of most cancer cells. This means that chemotherapy also harms cells that divide rapidly under normal circumstances. Most chemotherapeutic drugs work by impairing mitosis (cell division), effectively targeting fast-dividing cells and inducing apoptosis.

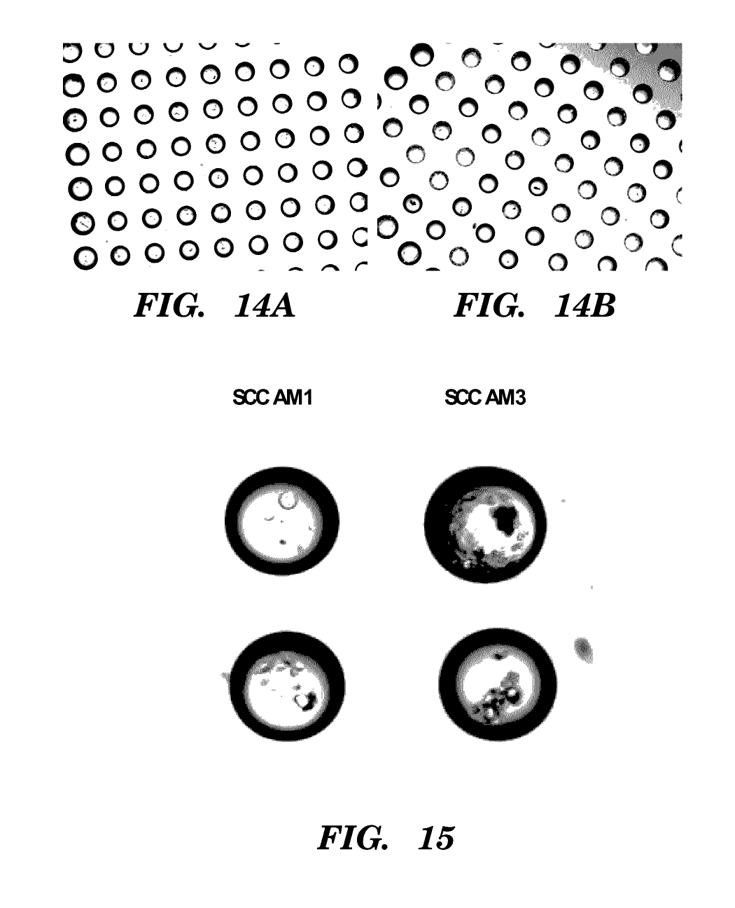

When SCC cells were seeded into arrays for 7 days, distinct clonal morphologies were generated as shown in FIGS. 13A and 14A. After Day 7 the cell culture media was changed and 10 uM or 10 nM cisplatin and incubated for 2 to 7 days, respectively. After which the chips were transferred into fresh media prior to a live/dead staining with CalceinAM/PI followed by stem cell marker analysis. Results shown in FIGS. 13B and 14B demonstrate a strong correlation between distinct cell population and chemo drug resistance; the spheroid cells have greater resistance in response to chemo drug than the spread cell. This confirms that the sphere cells maybe quiescent and non-dividing; in opposite, SCC spreading cells are rapidly growing and dividing. Thus, cisplatin can be used for spheroid enrichment and to test for enhanced expression of cancer stem cell markers.

### Example 4

### Identification of Tumor Initiating Cells by Surface Stem Cell Markers

The unique structure of MBs allows researchers to dissect the heterogeneity of tumor samples at the single cell level to evaluate its proliferative potential and to develop tumorigenic markers. This example takes advantage of the unique characteristics of MBs for the isolation, characterization, and in situ identification of specific markers for metastasis and novel targets for cancer chemotherapy. Antibody staining with two commonly used stem cell markers CD44 and CD133 was performed to investigate the cancer stem cell population in the established SCC cell lines. CD44 and CD133 are two commonly used stem cell markers. CD44 is a large cell surface glycoprotein that is involved in cell adhesion and migration and is one of the most well-known markers for cancer stem cells. A number of studies have suggested that CD133+ cells isolated from head and neck squamous cell carcinoma cell lines display increased clonogenicity, tumour sphere formation, self-renewal, proliferation, multilinear differentiation, and tumorigenicity.

Cells were cultured in MBs for up to 7 days after establishing different morphologies. Cells were first stained with CalceinAM for 45 minutes, a widely used reagent for determining cell viability. Chips were washed by submerging in PBS for 30 seconds in the incubator. Cells in the MB arrays were fixed by moving the chips into a well containing 10% formalin and 0.1% TritonX100 in PBS. The chips were incubated for 20 minutes under room temperature and were washed twice with PBS (5 minutes each wash under room temperature). 2% BSA was pipetted onto the top of the chip to block the surface for about 30 minutes under room temperature followed by another wash with PBS. Primary antibody solutions were prepared using 1/500 dilution of each antibody in 2% BSA/PBS. The primary antibodies used in chemo drug resistance experiments were Rabbit-Anti-PROM1/CD133 and Mouse-Anti-CD44. The chips were then transferred into 1 ml of primary antibody solutions and were incubated overnight at 4° C. or for 1 hour at room temperature. The chips were then washed twice with PBS before staining with secondary antibodies. The secondary antibody solutions were prepared using 1/500 dilution of each antibody in 2% BSA/PBS. The secondary antibodies used in chemo drug resistance experiments were Goat-Anti-Rabbit IgG (Texas Red conjugated) and Goat-Anti-Mouse IgG1 (CF350 conjugated). After wash, the chips were transferred to the secondary antibody solution and were incubated for 1 hour followed by two washes with PBS. The chips were imaged using a fluorescent microscopy.

In a second stem cell marker staining experiment, it was investigated whether Cisplatin has the ability to enhance expression of cancer stem cell markers. After cells establish distinct cell morphologies in MBs (˜7 days), 10 μM of Cisplatin was introduced to the culture media for 2 days. Chips were transferred to fresh media and were cultured for another 3 days before staining. The primary antibodies used in this experiment were Rabbit-Anti-PROM1/CD133 and Mouse-Anti-CD44. The secondary antibodies used were Goat-Anti-Rabbit IgG (Texas Red conjugated) and Goat-Anti-Mouse IgG1 (CF350 conjugated).

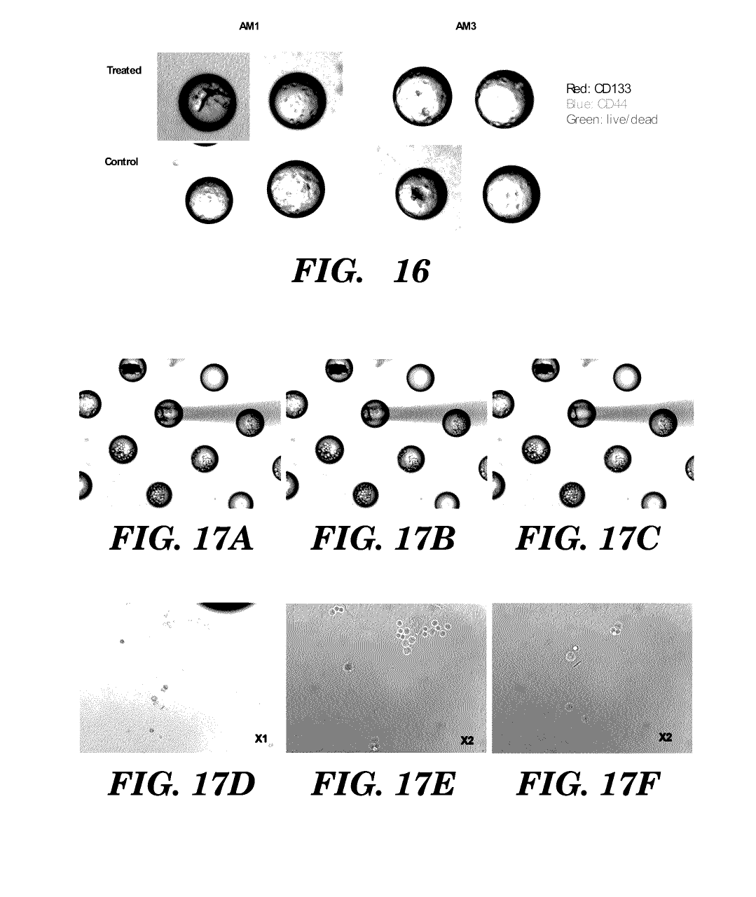

Results showed some CD133+ and CD44+ wells with low level of green fluorescent live cells in the treated chips (see FIG. 16). However, most cells were negative for both CD44 and CD133 proving that TICs is rare in the cell population, which is expected. The results shown in FIG. 12 demonstrate that spheroid cells in the SCC cell population appear to be CD44 and K5 positive. It was calculated that 39.5% of spheroid cell population stain positive for CD44 alone, 39.5% of spheroid cell population stain positive for CD133 alone, and 34.2% of spheroid cell population stain double positive for both CD44 and CD133. The results suggest that ˜⅓ of cells with spheroid morphology express both cancer stem cell markers, which verifies the strong correlation between spheroid cell population and TICs.

### Example 5

### Cell Recovering from MBs Using Micromanipulation

To enable off-chip proliferation and characterization of sorted cells, they must be recovered from the MB wells. MBs chips placed in 24-well TCP were transferred into wells containing serum free DMEM media and rinsed two times, then incubated for 10 minutes in the serum free media. To revoke adherent cells, the MBs chips were then submerged into 0.25% trypsin-EDTA solution and incubated for 30 minutes to loosen cells prior to aspiration (for nonadherent clones trypsin will not be needed). The chips were then transferred to 35 mm TCP with growing media and 10% FBS. Micromanipulation tools were used to recover and culture cells from different morphologies. Recovered cells was plated initially into 96-well TCP, and later expanded into larger wells. The diameter of SCC spread cells were around the inner diameter of the pipette tips (˜15 μm). The diameter of SCC spheroid cells were around 30-40 μm. Microcapillaries with proper inner diameter are required to recover cells with different sizes. Characterized clonal cell population from MBs could then be selected and recovered (see FIG. 17).

### Example 6

### Correlating the in vivo Metastatic Potential of A375 Melanoma Cells with in vitro Growth Characteristics

The metastatic potential of cancer cells is an elusive property that is indicative of the later stages of cancer progression. The ability to distinguish between poorly and highly metastatic cells is invaluable for the effective diagnosis and treatment of cancer. The microbubble architecture was utilized to differentiate among three A375 melanoma cell line derivatives (A375.P, A375.MA1, and A375.MA2) that vary in metastatic potential based on their growth characteristics including morphology and migratory potential. These cell lines (A375.P, A375.MA1, and A375.MA2) were produced via an in vivo selection process in which they were respectively shown to have increasing aggressiveness or metastatic potential in terms of tumor formation rate, size, and number. Gene array studies confirmed a signature of metastatic aggressiveness with expression of many genes encoding secreted and membrane proteins important in orchestrating interactions within the tumor-microenvironment including the endothelin receptor and the Frizzled homolog 7 receptor.

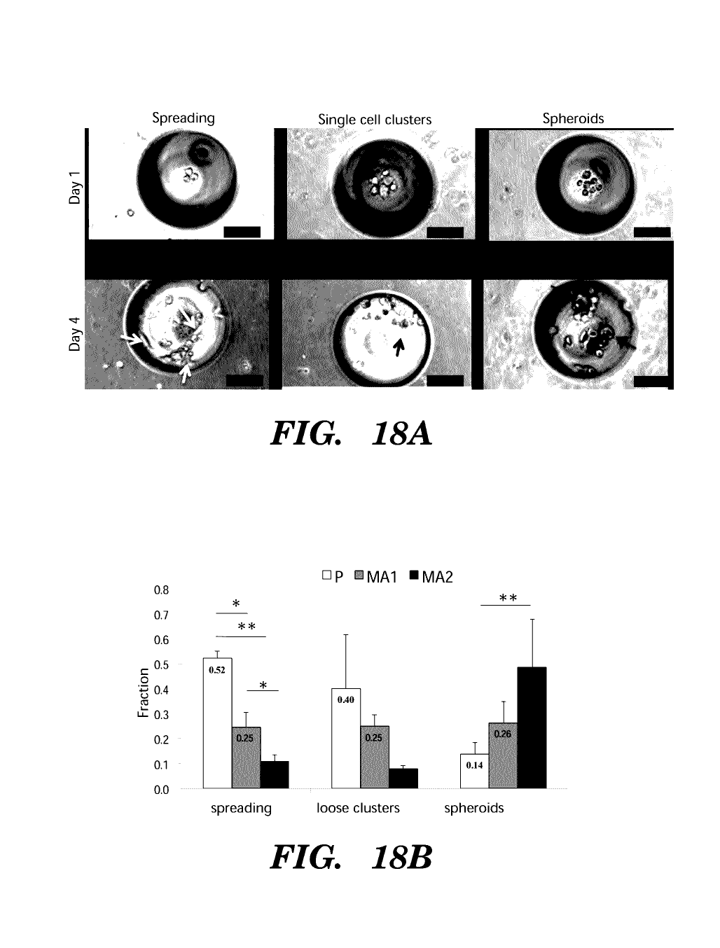

When these melanoma cells were cultured in MB wells they were observed to proliferate with three distinct colony morphologies classified as spheroid, clustered, and spreading (see FIG. 18A). Spheroid cultures display a large number of cells in a tight cluster with undefined cell boundaries. The clustered morphology showed multicellular clusters that are composed of easily identifiable individual cells. Spreading morphology contained elongated flat cells. Analysis of the frequency of each morphology type (see FIG. 18B) showed that the spreading morphology appeared highest in the poorly metastatic cells (A375.P) and least in the highly metastatic A375.MA2 cells (p<0.005).

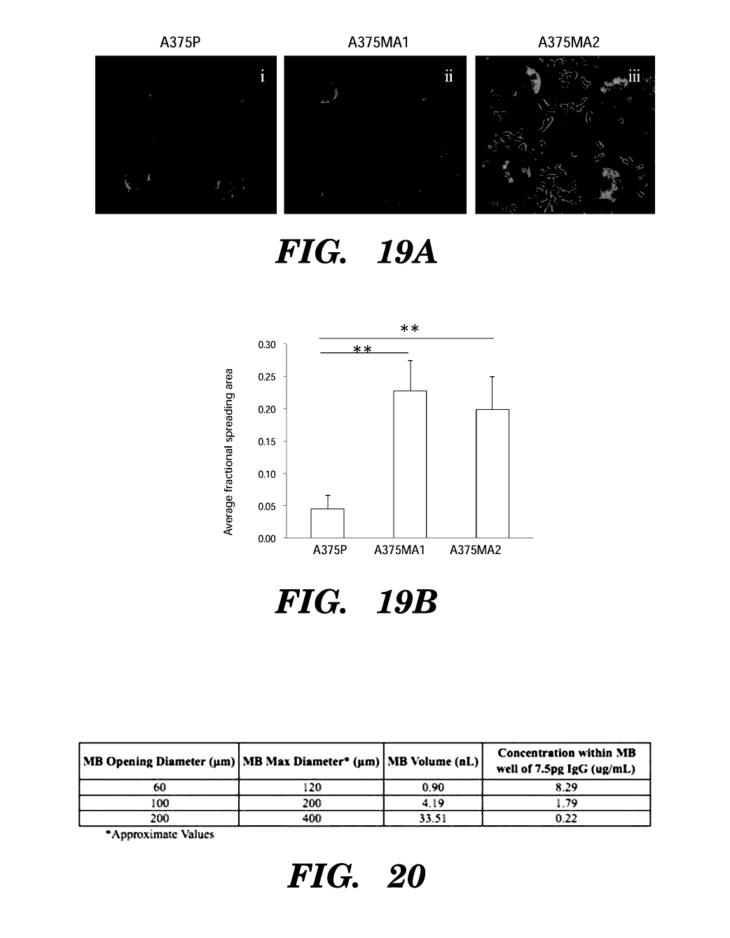

Metastatic cells have an increased success rate of dissemination from the primary tumor to a secondary site compared to the majority of cancer cells. The ability of these cells to undergo the epithelial-mesenchymal transition (EMT) was demonstrated utilizing the microbubble radial migratory assay. The radial migration of the A375 cell series from MB wells was monitored over a 6 day period. Microbubble wells examined for this experiment were each initially seeded with 10-20 cells per MB well. Results show that the A375.MA1 and A375.MA2 cells migrate out of the MB well onto the flat PDMS chip surface and adopt a spread morphology whereas by day 6 the A375.P cells remain confined within the MB well exhibiting a more round appearance (see FIG. 19A). The source of the cells proliferating on the chip surface was from the MB well as rigorous washing were performed at the time of cell seeding to remove loose cells from planar chip surface.

At day 6 the area of cell spreading on the planar PDMS chip surface was quantified (see FIG. 19B). The average fractional area of cell spreading from the MB wells is 0.05±0.02, 0.23±0.05, and 0.20±0.05 for the A375 P, A375 MA1 and A375 MA2, respectively. These results were from three sets of data with each set containing four top-view images of MB wells focused at the opening of the MBs. Using the one-tailed Student's t-test, the difference between the A375 P and both A375 MA1 (p=1.43e-6) and A375 MA2 (p=3.4e-5) was found to be statistically significant. However, there was not a statistically significant difference in migration between A375 MA1 and A375 MA2. The rationale for this lack of a difference is the intermediary characteristics and cellular heterogeneity of A375 MA1 resulting from the in vivo selection process (Xu et al., Molecular Cancer Research: MCR 6:760 (2008), which is hereby incorporated by reference in its entirety).

The results of the radial migration assay was also compared to the well-known scratch recovery assay, which measures the ability of cells cultured on TCP to proliferate, migrate and fill the gap created by a scratch on the TCP surface using, e.g., a pipette tip. After Day, the scratch recovery percentages for A375 P, A375 MA1, and A375 MA2 were 29.6±8.2%, 25.7±5.0% and 48.1±4.0%, respectively. A375 MA2 consistently showed the most rapid migration of both leading edges of the scratch, showing statistically significant differences compared to the A375 P (p=0.0047) and A375 MA1 (p=0.00008). These migration results are not due to proliferation differences between the cell lines. Results from A375 P and A375 MA1 also show recovery by Day 3; however, there was no significant difference between these two cell lines (p=0.251), which may reflect the increased heterogeneity of the A375 MA1 compared to the A375 MA2 resulting from the in vivo selection process (Xu et al., Molecular Cancer Research: MCR 6:760 (2008), which is hereby incorporated by reference in its entirety).

The recovery rate of the A375 MA1 cells in the scratch assay was more similar to the poorly metastatic A375 P line. However, in the radial migration out of the MB wells on Day 6, the intermediate A375 MA1 cell line shows more similarities to the highly metastatic A375 MA2 line. It is also observed that the A375 MA2 cells appear more spread than the A375 MA1 cells.

These results demonstrates that the highly metastatic A375.MA1 and A375.MA2 lines have a higher frequency of the spheroid morphology and a distinctive radial spreading growth pattern out of the microbubble well, which indicates a selection for highly migratory cells. Spheroid morphology in other cancer cell types (Doillon et al., Anticancer Research 24:2169 (2004); Kenny et al., Molecular Oncology 1:84 (2007); Kim et al., Breast Cancer Research and Treatment 85:281 (2004); Valcarcel et al., J Translational Medicine 6:57 (2008); Wang et al., Seminars In Cancer Biology 15:353 (2005), each of which is hereby incorporated by reference in its entirety) and in melanoma (Chandrasekaran and DeLouise, Biomaterials 33:9037 (2012); Kwok et al., Cancer Research 49:3276 (1989); Schatton and Frank, J. Investigative Dermatology 130:1769 (2010), each of which is hereby incorporated by reference in its entirety) has been correlated with a more aggressive cell phenotype, which is consistent the results obtained here. This is the first example, however, where the heterogeneity of a cell line can be correlated with metastatic potential without use of special defined media to induce sphere formation.

In addition, A375.MA1 cells showed the highest tendency to proliferate from a single cell. In future studies, specific spheroid cells can be targeted and recovered to monitor self-renewal, proliferation, phenotypic characterization, and genetic profiling utilizing the novel MB array technology. Clonal cell populations can be selected and recovered from MBs. Invasion assays and mouse studies can be used to perform genetic profiling and functional testing on characterized clonal cell populations.

### Example 7

### Assessing Clonogenic Potential of A375 Melanoma Cells

Typically, clonogenic potential is measured by seeding cells in a 96-well plate under limiting dilution conditions; plating 0.2 to 20 cells per μL or 20 to 2000 cells per well (Pastrana et al., Cell stem cell 8:486 (2011), which is hereby incorporated by reference in its entirety). In this Example, the MB array is instead used to measure the clonogenic potential of single cells seeded into individual wells. The MB wells used for these measurements had a diameter of 160 μm and a volume of ˜3 nL. It has previously been shown that the number of cells seeded into the MB cavity array is dependent on the stock concentration and incubation time of the cell suspension (Chandrasekaran and DeLouise, Biomaterials 32:9316 (2011), which is hereby incorporated by reference in its entirety). For seeding single cell cultures a stock concentration of 1×10⁴ cells/mL (or 1×10³ cells/cm²) was used. 50 μL of the stock solution was applied onto the top of the MB chip for 5 min in a humidified chamber. Next, the solution was removed and the chip was rinsed twice with 50 μL media (DMEM+10% FBS+1% P/S). Then the chip was transferred via sterile forceps to a well of a 24-well TCP that contained 1 mL of media. It was then shaken laterally to remove any excess cells that may have remained on the surface. This was repeated twice. Lastly, the cell seeded MB chip was transferred to a new well containing 1 mL of new media. Cell media was exchanged every 3 to 4 days.

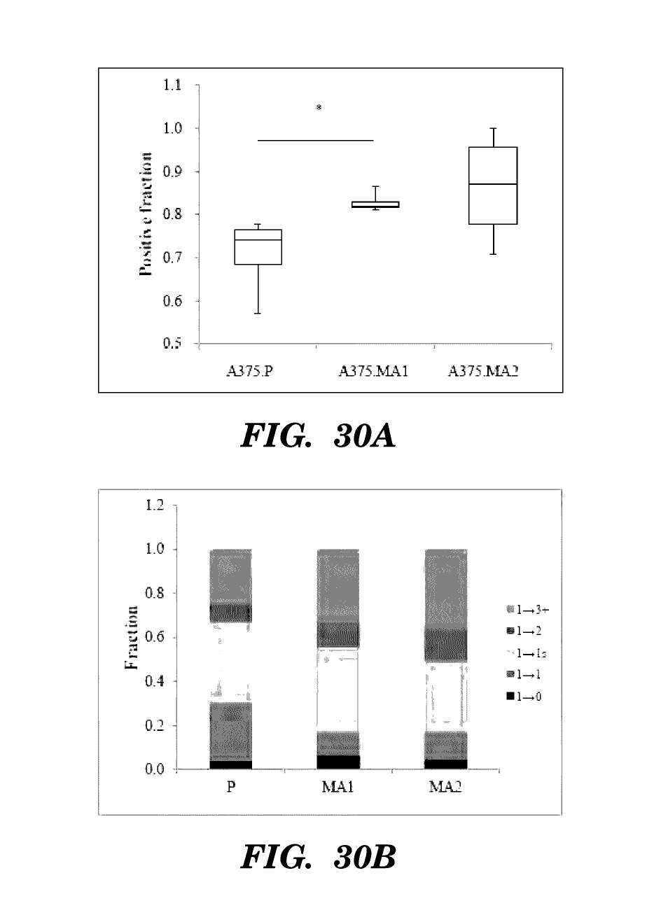

For each cell line, two 10×10 MB arrays were analyzed on a 1.0 cm×0.5 cm chip. Images were taken 2 h after seeding to establish the single cell seeding efficiency. The images taken were focused primarily on the bottom of the MB cavity. Due to the curvature of the MB, seeded cells settle at the bottom of the MB cavity. Due to the stringent washing steps that were done to remove cells deposited onto the chip surface, there were few to no cells that could be found on the flat PDMS surface following the MB seeding protocol. After Day 4 or 5, the MBs were reimaged to obtain the final count of cells in the MB cavities. These were tabulated and the wells were labeled either positive or negative. Positive data referred to MB wells that were seeded with a single cell and showed either an obvious increase in cell number (proliferation) or cell size (sphere with >50% increase in the diameter of the cell). Negative data refers to singly seeded MB well that showed dead cells or a lack of proliferation by Day 5. In addition to quantifying the positive fraction of wells seeded with a single cell, the following categories were also quantified: 1→1s (1 sphere cell, >50% the size of a single cell), 1→2 cells, 1→3+ cells. See FIG. 30B.

The capability a cell to clonally proliferate is a measure of its ability to overcome stringent environmental conditions and thrive (Chandrasekaran and DeLouise, Biomaterials 32:9316 (2011); Nomura et al., Cancer Research 49:5288 (1989); Yin et al., European J Cancer 29A:2279 (1993), each of which is hereby incorporated by reference in its entirety). A high clonogenic potential is expected to be a characteristic of a cell with high metastatic potential (Chandrasekaran and DeLouise, Biomaterials 32:9316 (2011), which is hereby incorporated by reference in its entirety). Metastatic cells that extravasate to a secondary site must have the ability to condition their microenvironment in order to expand to form a tumor mass (Dvorak et al., J Surgical Oncology 103:468 (2011); Laconi, Bioessays: News and Reviews in Molecular, Cellular and Developmental Biology 29:73 8 (2007); Mbeunkui and Johann, Cancer Chemotherapy and Pharmacology 63:571 (2009); Weber and Kuo, Surgical Oncology 21:172 (2012); Whiteside, Oncogene 27:5904 (2008), each of which is hereby incorporated by reference in its entirety).

In this Example, the microbubble well array was used to investigate the capacity of the A375 cell lines to undergo clonal expansion. The small media volume per cell (3 nL of media/cell) and the unique spherical MB well architecture allows for rapid conditioning of the microenvironmental through the concentration of factors produced by the cell. In this assay, cells were not given an adherent matrix to make the culture system more stringent. True clonality can be guaranteed only by plating one cell per well, which is achieved by adjusting the MB well array cell seeding density. The array format enables analysis of many clones simultaneously. Results obtained from analysis of single cell cultures of the three A375 melanoma derivatives: A375 P, A375 MA1, and A375 MA2 are depicted in FIGS. 30A-B. Two MB well arrays were evaluated for each cell line and each array consisted of 100 MB wells. The total number of single cell cultures examined for A375 P, A375 MA1, and A375 MA2 cell lines were n=73, 90, and 89, respectively. The positive fraction is defined as the number of MB wells seeded with a single cell that showed either clonogenic expansion (an increase in cell number). The A375 P, A375 MA1 and A375 MA2 cell lines showed average positive fractions of 0.71±0.05, 0.83±0.01, and 0.86±0.07, respectively. See FIG. 30A. Statistical analysis showed a significant difference in the response between the A375 P and A375 MA1 (p=0.039). There was no statistically significant difference between A375 MA1 and A375 MA2 (p=0.326), but there was nearly a statistically significant difference between the A375 P and A375 MA2 cell lines (p=0.056). An in-depth examination of the distribution of single cell proliferation outcomes reveals distinct differences in the positive clonogenic responses between the A375 P and A375 MA2 cell lines. For example, it was observed that the fractional % of positive single cell cultures counted with high proliferation (1→2 and 1→3+) was higher for the highly metastatic cell line A375 MA2 (50.5%) compared to the poorly metastatic cell line A375 P (32.8%). See FIG. 30B. Further, the A375 P cell line showed a markedly higher frequency (30.1%) of cells that did not proliferate (1→1) compared to the A375 MA2 line (16.8%). See FIG. 30B. These results suggest that the more metastatic cell lines (A375 MA1, A375 MA2, have higher proliferative potential than the poorly metastatic A375 P melanoma cell line in MB culture.

### Example 8

### Optimizing Well Size to Maximize Single Cell Sorting Efficiently

To efficiently sort cells by what they secrete it is important to optimize the MB well geometry. According to an embodiment, the time to observe signal generation scales microbubble cavity size. The smaller the microbubble cavity, the smaller the volume and the quicker the concentration of secreted product rises to detectable level. During the evaluation of the effects the size of the MB well has on its fluorescent signal development and single cell assay properties, an experiment was performed using SA13 cells seeded into MB arrays that had been coated with an IgG capture coating and then imaged every day for 7 days to monitor cell growth and IgG detection within the MB wells. It was hypothesized that the use of smaller MB wells would allow for the concentration of secreted factors quicker and thus could be detected quicker by fluorescence microscopy. FIG. 20 shows the approximate sizes of each of the MB wells (referred to as 60 μm, 100 μm, and 200 μm MB wells) and in the far right column of the graph shows the estimated calculated concentrations within the MB wells, not taking into account any changes in diffusion between the MB well sizes. It has been estimated that SA13 cells secrete about7.5 pg of IgG per cell per 24 hours. Thus, given this mass, assuming 1 cell per MB well, the concentration in a 60 μm MB well would reach 8.29 μg/mL within 24 hours, whereas in a 100 μm MB well the concentration would be just 1.79 μg/mL, and for a 200 μm MB well would only be 0.22 μg/mL. This calculation also does not take into account any differences in diffusion rates between the MB well with the bulk media. Although the 200 μm MB wells have a deeper well, they also have a large opening, which could allow quicker diffusion between the MB well and bulk media and even lower concentrations of cell secreted factors within the MB well.

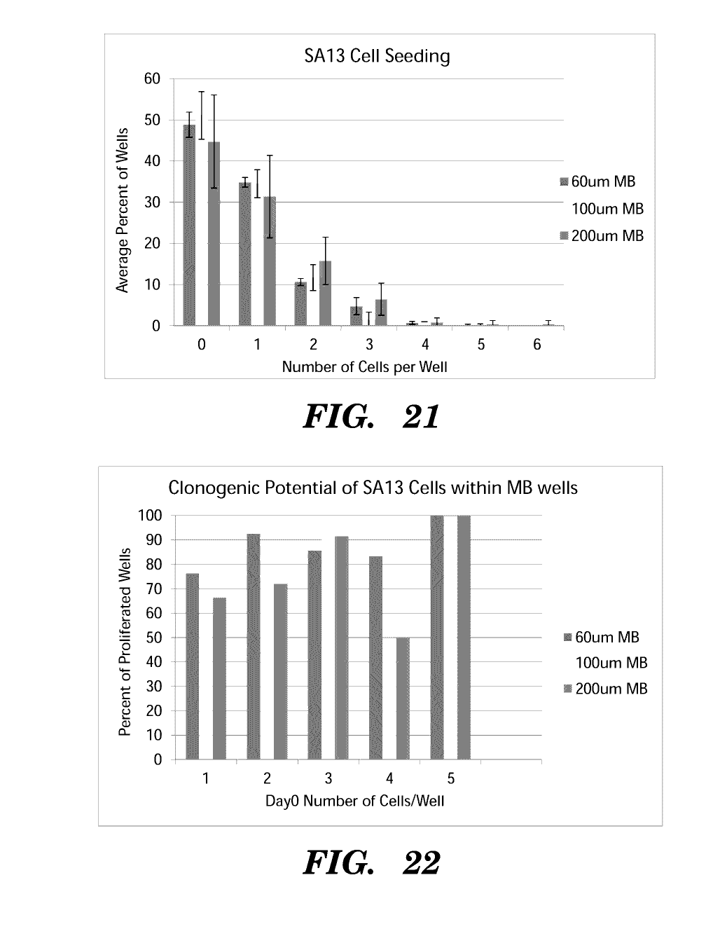

Due to the differences in the MB opening diameter size between the 60, 100, and 200 μm MB wells, the cell seeding densities had to be varied during the MB seeding process to account for the differences in MB cell capture. FIG. 21 shows a graph of the cell seeding statistics for the MB wells of varying size. The 60 μm MB wells were seeded using a cell density of 2,000 cells/cm², the 100 μm MB wells at 5,000 cells/cm², and the 60 μm MB wells at 10,000 cells/cm² in order to achieve similar MB cell seeding statistics between the three sizes. The larger 200 μm MB wells tended to have slightly more 2 or more cells per MB well, this is due to the larger diameter MB opening which allows for more area for cells to fall into the MB wells.

Once the cell indexing was done after seeding the MB wells, the cells were allowed to culture in the MB chips for 4 days and then counted again to track the cells that had gone on to proliferate. The MB wells that started with 1, 2, 3, 4, or 5 cells per well were determined which had proliferated and then the percent of each group calculated to determine the clonogenic potential. FIG. 22 shows the graph of the average clonogenic potentials per MB well size. The 60 μm MB wells had better clonogenic potential for wells seeded with 1 or 2 cells per well than the larger MB wells. This could be due to the smaller size of the wells allowing the cell-secreted factors to concentrate in the MB well quicker and thus act as a better-conditioned niche environment for the single cells to grow and proliferate.

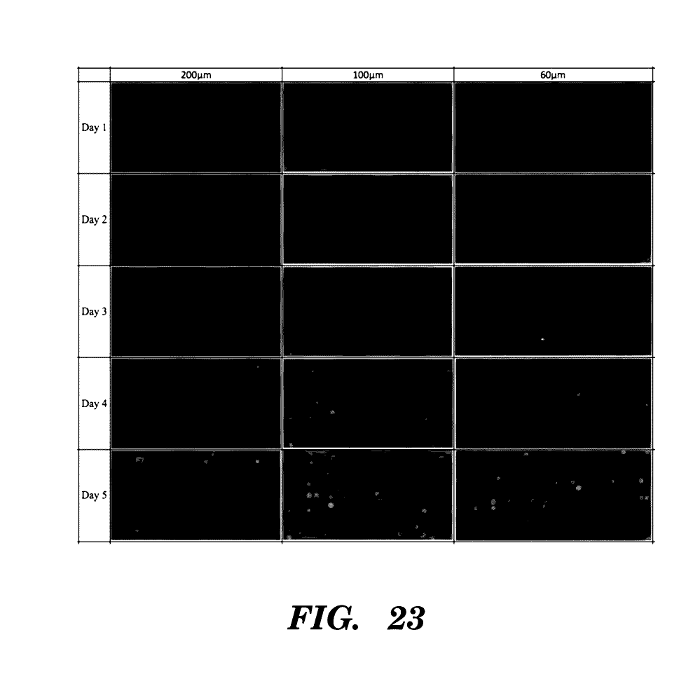

The microscope fluorescence images, all taken under the same imaging set-up and exospore time, were equally enhanced using the ImageJ brightness/contrast threshold feature. Images from days 1 through 5 post-seeding were compared for fluorescent ring development. As shown in FIG. 23, positive MB fluorescent rings can begin to be seen by eye on day 2 in the 60 μm MB wells, but one doesn't begin to see fluorescent rings in the 100 μm MB wells until about day 3 and not until about day 4 for the 200 μm MB wells.

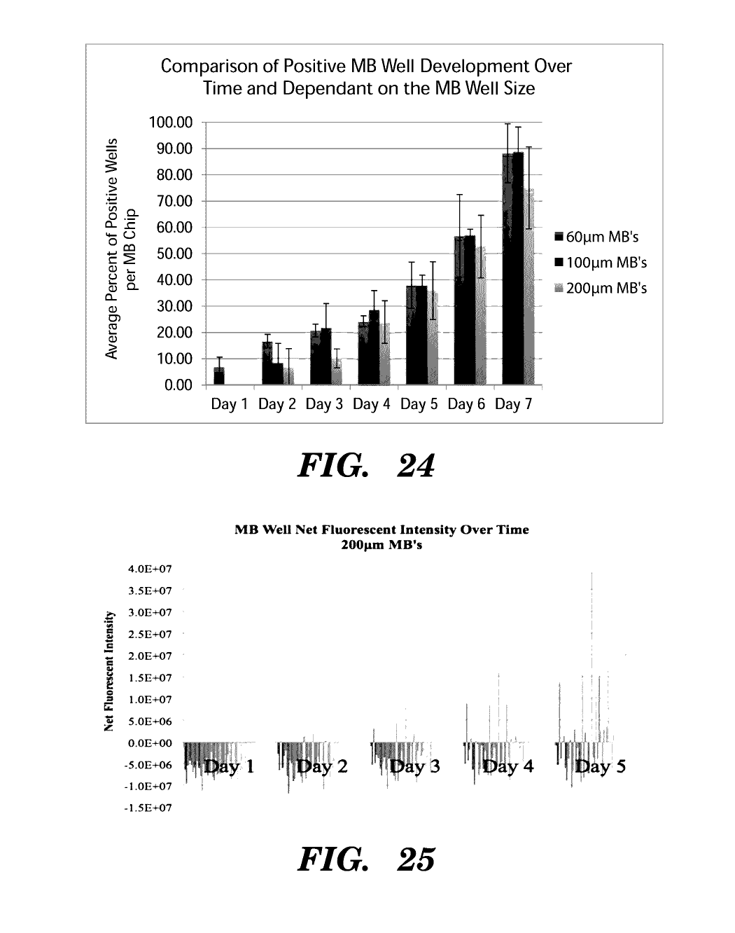

The microscopy images were also analyzed using the Matlab program developed at the University of Rochester Medical Center. An average of the negative MB wells per each MB size group on day 1 was used as a baseline for the negative MB value. To determine the limit for positive MB wells, a value of 3 times the standard deviation was added to the average negative value. Any MB fluorescent signal values above this baseline value were then determined to be a positive MB well. In the graph below in FIG. 24, the average percent of positive wells seen on the MB chips per size group were compared for each day. By the end of day 4, the cells had begun proliferating out of the MB wells and were growing on the MB chip surfaces and spilling over into other MB wells. This is why on days 5-7 a large spike in the percent of the wells that are positive is seen.

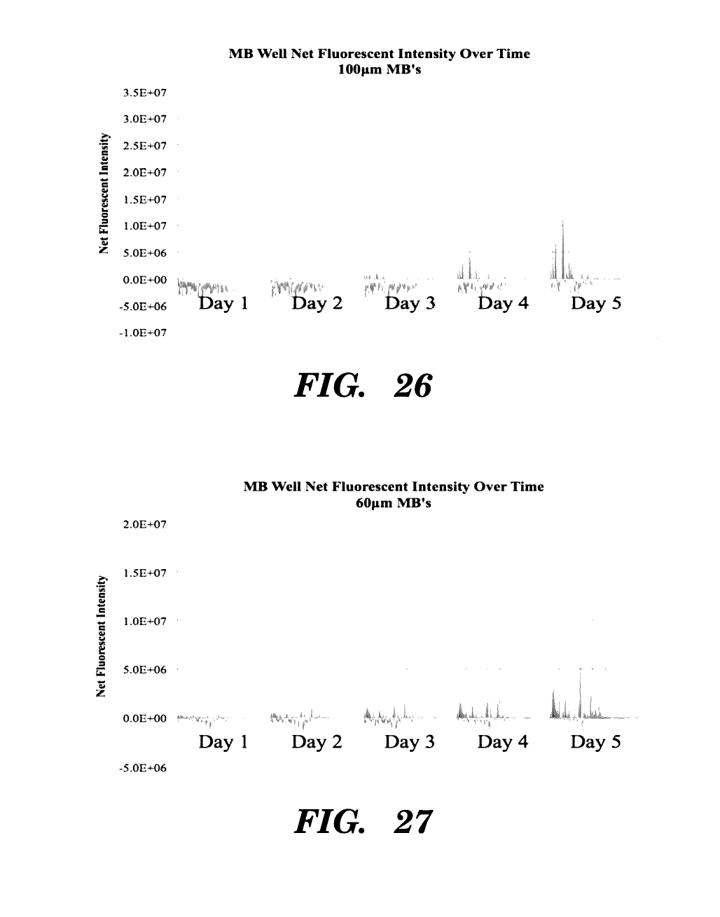

Graphs were also prepared comparing every MB analyzed per group and its fluorescent signal over the course of 5 days. FIGS. 25, 26, and 27 show the fluorescent development in the 200 μm, 100 μm, and 60 μm MB wells tracking individual MBs. The threshold level for a positive MB well for 200 μm is a fluorescent intensity value above 153,551; for 100 μm MB wells it needs a positive fluorescent intensity value above 314,261; and for 60 μm MB wells a positive fluorescent intensity value must be above 675,067. In FIG. 25, one can see that not many positive fluorescent MB wells start showing up until day 4, whereas in FIG. 26 once can see positive MB wells on day 3, and in FIG. 27 many positive MB wells on day 2 can be seen.

### Example 9

### Sorting Cells by Secretion Rate

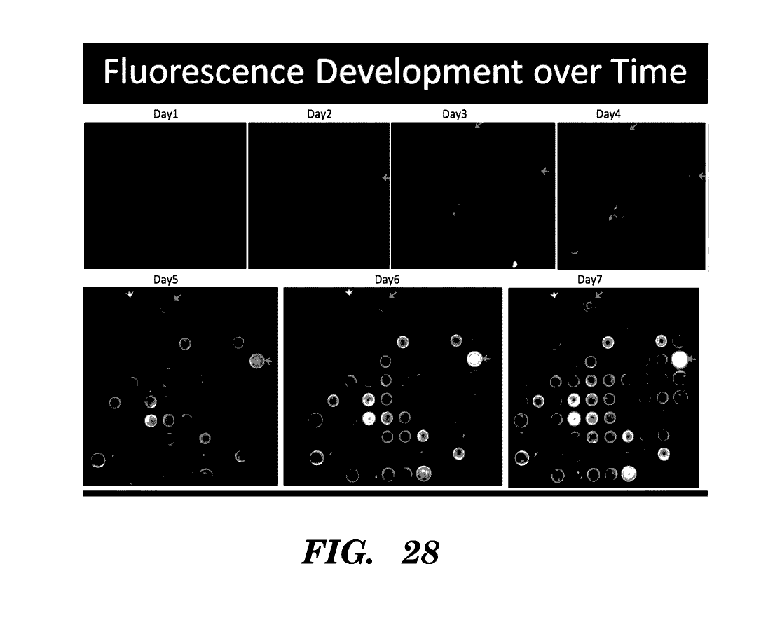

In one embodiment, the present invention includes a single cell screening method using MB well arrays to sort cell by secretion rate. It is recognized by those skilled in the art that hybridoma or transfected CHO cells that are engineered to secrete antibodies to a specific target may produce antibody at different rates. For efficient manufacture of monoclonal antibody therapeutics it is desired that highest secreting cells be discovered. Using the methods disclosed above and taking advantage of the ability to culture cells from hours to days to weeks it is possible to dissect the heterogeneity of cell by their secretion rates simply by monitoring the development of signal over time. Microbubble wells containing cells that secrete at a high rate will develop signal within 2-24 hours whereas slow secretors will develop positive signal in 3 to 4 to 7 days (see FIG. 28).

### Example 10

### Sorting Cells by Functional Assays

The invention should not be construed as being limited solely to a device and methods for antibody discovery based on antigen binding. Functional assays that take advantage of the microbubble well architecture and small nanoliter volume that allow cells to condition their microenvironment quickly with secreted factors, the low elastic modulus which is more similar to in vivo mechanical properties rather than polystyrene tissue culture plastic, and the ability to culture cells over a period of time allow functional assay screens. The ability to culture cells over time allows for selection of cell by secretion rate. It also allows for screening functional assays to be designed based on cell migration, signaling pathways, cell-cell interaction, pathogen or toxin neutralization, antibody dependent cellular cytotoxicity, antibody mediated tumoricidal activity, or opsonization activity.

One skilled in the art would appreciate, based upon the disclosure provided herein, that cells producing antibodies can be screened based on their ability to bind peptide epitopes, nanoparticles, surface receptors, or target antigen. The present invention is not limited to using the microbubble well device and methods to screen for antibodies. Rather, the present invention includes using the device and methods to screen for cancer stem cells, to screen cytokines, cell surface molecules, or other factors produced by cells. The cells for screening that produce antibodies or cytokine or other secreted factors can be produced by immunizing an animal such as, but not limited to, a rabbit, a mouse, a rat, or a camel, with an antigenic particle of the invention, or a portion thereof, by immunizing an animal using a composition comprising at least a portion of the antigen, a complex molecule, a cell, a cell fraction, or a fusion composition including a tag polypeptide portion comprising, for example, a maltose binding protein tag polypeptide portion, covalently linked with a portion comprising the appropriate amino acid residues. One skilled in the art would appreciate, based upon the disclosure provided herein, that cells be primary B cells, or hybridoma, or stable or transiently antibody expressing cell lines (e.g. CHO, HEK293 cells). Assays may also seek to screen cells based on secretion rate, morphology, proliferation or drug resistance.

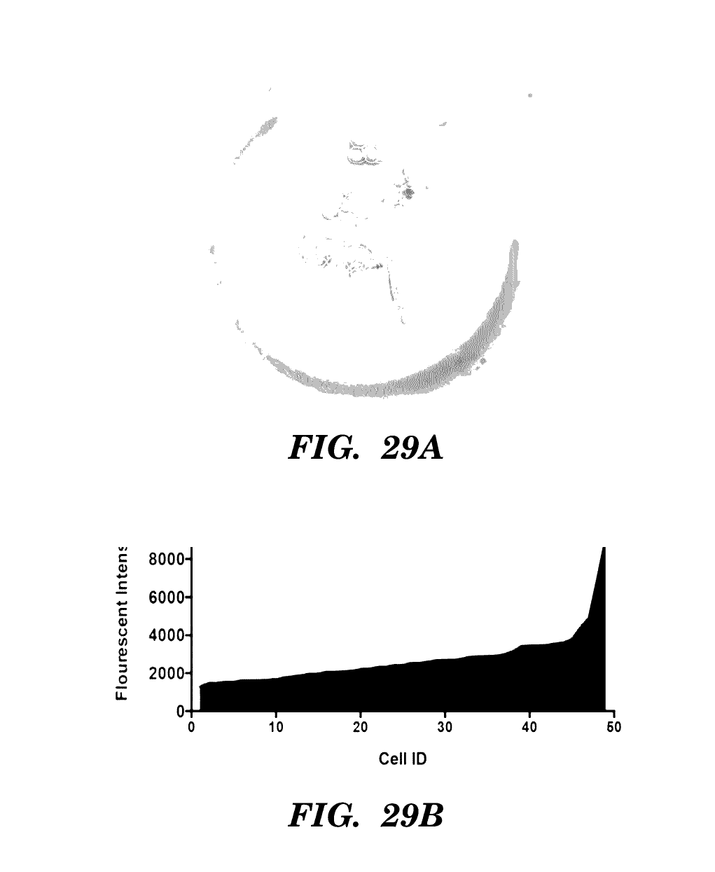

The microbubble opsonization assay relies on antibody tagging of bacteria to be targeted for phagocytosis by macrophages. Once engulfed by the macrophage, the phagosome fuses with a lysosome and the tagged bacteria is lysed/destroyed through the use of peroxides and digestive enzymes. Simplistically, microbubble arrays are seeded with B cells, incubated to allow antibody to accumulate within the microbubble well, and then activated macrophage cells are added into the MB array in the presence of fluorescent reporter-tagged bacteria. MB well containing B cells producing opsonizing Ab can then be identified by observing macrophages containing fluorescently labeled bacterial particles, such as seen in FIG. 29. The general procedure is that bacteria is labeled using the pHrodo™ amine reactive labels (Life Technologies Corp., Carlsbad, Calif.). These fluorescent dyes are quite helpful since they do not fluoresce until they encounter a change in pH within the phagosome after they have been internalized. B cells are seeded into the microbubble array to maximize the number of MB wells with single cells and cultured to proliferate and to accumulate secreted Ab. To screen mouse B cells, the murine macrophage cell line RAW264.7 cells could be used and to screen human B cells the human monocyte/macrophage cell line u937 could be used. The macrophage cell lines are seeded into MB arrays and allowed to settle into MB wells before media is replaced with pHrodo™ labeled bacteria, and then MB arrays incubated and imaged. Due to the morphological and fluorescent resolution of microbubbles, the requirement of using pHrodo labeled bacteria may not be absolute and any suitable reporter tagged bacteria may be sufficient to resolve opsonization.

The invention encompasses discovery of sorting single cells including tumor initiating cells and cells that secrete antibodies for drug discovery and personalized medicine. Drugs include monoclonal, synthetic antibodies, and the like. One skilled in the art would understand, based upon the disclosure provided herein, that the crucial feature of the invention is that the antibody bind specifically with an antigen of interest. That is, the antibody of the invention recognizes an antigen of interest or a fragment thereof (e.g., an immunogenic portion or antigenic determinant thereof), causing immunoprecipitates or signal using affinity capture with fluorescently labeled antigen or antigen coated fluorescent beads which are standard methods well-known in the art.

One skilled in the art would appreciate, based upon the disclosure provided herein, that cells producing antibodies can be screened based on their ability to bind peptide epitopes, nanoparticles, surface receptors, or target antigen. The present invention is not limited to using the microbubble well device and methods to screen for antibodies. Rather, the present invention includes using the device and methods to screen for cancer stem cells, to screen cytokines produced by immune cells. The cells for screening that produce antibodies or cytokine or other secreted factors can be produced by immunizing an animal such as, but not limited to, a rabbit, a mouse or a camel, with an antigenic particle of the invention, or a portion thereof, by immunizing an animal using a composition comprising at least a portion of the antigen, or a fusion composition including a tag polypeptide portion comprising, for example, a maltose binding protein tag polypeptide portion, covalently linked with a portion comprising the appropriate amino acid residues. One skilled in the art would appreciate, based upon the disclosure provided herein, that cells be primary B cells, or hybridoma or CHO cells. Assays may also seek to screen these cell based on secretion rate, morphology, proliferation or drug resistance.

### Example 11

### Development, Analysis, and Isolation of Micro-Organs, Microtissues, and Embryoid Bodies

The unique architecture and long-term single cell initiating culture ability of the microbubbles enables their utility for stem cell and tissue generation applications. These include but are not limited to the culture, selection, and differentiation of stem cells from different sources and their development into micro-tissue, micro-organ structures, and organized multicellular architectures. These structures can be screened using microbubbles for morphology, secreted product, cellular composition, and functional activity.

### Example 12

### Microbubble Array-Based Molecular Biology Reactions

The thermodynamic properties of PDMS, nano-liter volume, and high-throughput capacity of the MB array can facilitate the performance of nano-scale molecular biology reactions. These include, but are not limited to on-chip polymerase chain reaction, CRIPSR-Cas9 and siRNA manipulation, screening and selection, in vitro protein translation, in vitro protein post-translational modification, in vitro polymerase activity screening, and nano-scale nucleotide sequencing.

### Example 13

### High-Throughput Cell-Based Drug Screening

The high-throughput and long-term culture capacity of microbubble arrays can facilitate the cell-based screening of compound libraries for cell-based outcome. This could include but is not limited to small molecule, peptide, protein, antibiotic, macrocycle-based compound library screening and lead optimization. Examples of outcome measures could include morphology, toxicity, cell signaling, or molecule secretion.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A method for analyzing a cell, the method comprising the steps of:
providing a microfluidic device, the microfluidic device comprising a substrate having: (i) a first surface; and (ii) a plurality of curvilinear cavities embedded in the substrate, each of the plurality of curvilinear cavities comprising an inner surface and an opening at the first surface to an exterior of the substrate, the opening having a first diameter, wherein the inner surface of each curvilinear cavity curves outward from a rounded bottom located at a point furthest from the opening of the cavity to a maximum diameter, and then curves inward from the maximum diameter to the opening at the first surface, the maximum diameter being greater than the first diameter;
adding a plurality of cells to the microfluidic device under conditions configured to allow at least some of the cells embed within one or more of the plurality of curvilinear cavities embedded in the substrate; incubating the microfluidic device under conditions suitable for the cells to survive for a first period of time; and
sorting the cells based on a first characteristic of the cells.

2. The method of claim 1, wherein the plurality of curvilinear cavities are configured to form a lattice, and further wherein the plurality of curvilinear cavities are spaced a predefined distance from each other.

3. The method of claim 1, further comprising the step of coating the plurality of curvilinear cavities prior to adding the plurality of cells.

4. The method of claim 3, wherein the coating comprises a chemical, biomolecule, or biochemical.

5. The method of claim 4, wherein the coating is selected from the group consisting of an antibody, a toxin, a growth factor, a selectin, a collagen, a fibronectin, a chemoattractant, a signaling molecule, an antigen, a ligand, a biochemical, and combinations thereof.

6. The method of claim 1, further comprising the step of coating the first surface with a cell or protein blocking agent.

7. The method of claim 6, wherein the coating is bovine serum albumin, casein, polyethylene glycol (PEG), or another blocking agent.

8. The method of claim 1, further comprising the step of adding a compound to the incubating cells.

9. The method of claim 1, further comprising the step of incubating the microfluidic device under conditions suitable for the cells to proliferate, wherein the conditions for survival and the conditions for proliferation may be identical or different.

10. The method of claim 1, wherein said first characteristic is selected from the group consisting of proliferation, morphology, drug resistance, adhesion, secretion rate, surface marker, ability to block a signaling pathway, ability to promote a signaling pathway, ability to enhance opsonization, and combinations thereof.

11. The method of claim 1, wherein the ratio of the maximum diameter to the first diameter is greater than 1.

12. The method of claim 1, wherein the maximum diameter is approximately 80 to 350 microns and the first diameter is approximately 40 to 200 microns.

13. The method of claim 1, wherein the cells are mouse hybridoma cells, CHO cells, B cells derived from human or animal peripheral blood or lymphoid organs, or cancer cells.

14. The method of claim 1, further comprising the step of detecting one or more secretions of the embedded cells.

15. The method of claim 14, wherein the secretion is detected with a fluorescently or chromogenic tagged antigen, peptide, cytokine, antibody or other protein or nanoparticle reporter.

16. The method of claim 1, wherein two or more different cell types are seeded.

17. The method of claim 1, wherein the substrate comprises a polysiloxane, a polyacrlyamide, a polyacrylate, a polymethacrylate, a carbon-based polymer or mixtures thereof.

18. A method for analyzing a cell, the method comprising the steps of:
providing a microfluidic device, the microfluidic device comprising a substrate having: (i) a first surface; and (ii) a plurality of curvilinear cavities embedded in the substrate, each of the plurality of curvilinear cavities comprising an inner surface and an opening at the first surface to an exterior of the substrate, the opening having a first diameter, wherein the inner surface of each curvilinear cavity curves outward from a rounded bottom located at a point furthest from the opening of the cavity to a maximum diameter, and then curves inward from the maximum diameter to the opening at the first surface, the maximum diameter being greater than the first diameter;
adding a plurality of cells to the microfluidic device under conditions configured to allow at least some of the cells embed within one or more of the plurality of curvilinear cavities embedded in the substrate;
incubating the microfluidic device under conditions suitable for the cells to survive for a first period of time; and
analyzing the cells.

19. The method of claim 18, wherein the plurality of curvilinear cavities are configured to form a lattice, and further wherein the plurality of curvilinear cavities are spaced a predefined distance from each other.

20. The method of claim 18, further comprising the step of visualizing one or more seeded curvilinear cavities.
